(19) United States
(12) Patent Application Publication
(10) Pub. No.: US 2005/0079598 A1
(43) Pub. Date: Apr. 14, 2005

(54) Apparatus and method for identification of biomolecules, in particular nucleic acid sequences, proteins, and antigens and antibodies

(76) Inventor: Randall W. Davis, Pleasanton, CA (US)

Correspondence Address: CHARLES LOUIS THOEMING, 1390 WILLOW PASS ROAD, SUITE 1020, CONCORD, CA, 94520, US

(21) Appl. No.: 10/681,630

(22) Filed: Oct. 8, 2003

(51) Int. Cl. C12M 1/34

(52) U.S. Cl. 435/287.1; 205/777.5

(57) ABSTRACT

An electrical detection system and method using an array of conductive sense sites within a sensing substrate for electrically detecting the successful hybridization or binding reaction between two chemical substances, particularly between biogenic substances such as nucleotides, proteins and ligands, and antigens and antibodies. The method and apparatus provide a an inexpensive, robust, small, repeatable, and intuitively easy to use apparatus for detection of low levels of hybridization with large numbers of closely spaced conductive sense sites within a single substrate.

### CROSS-REFERENCES TO RELATED APPLICATIONS

None.

### STATEMENT REGARDING FEDERALLY SPONSORED RESEARCH OR DEVELOPMENT

None.

### REFERENCE TO A MICRO-FICHE APPENDIX

None.

### BACKGROUND OF THE INVENTION

1. Field of the Invention

This invention relates to an array of sense sites for electrically detecting the successful hybridization or binding reaction between two chemical substances, particularly between biogenic substances such as nucleotides, proteins and ligands, and antigens and antibodies.

The use of microarrays has revolutionized the way that cellular processes are analyzed and have found widespread use in the laboratory including the study of: mRNA expression analysis; SNP (single nucleotide polymorphism) analysis; resequencing; whole genome copy number analysis; DNA-protein interaction; Protein-Protein interactions; and antibody-antigen identification. For the purposes of discussion, nucleic acid segments will be presented as the binding pair of molecules. However, this is just one example of the many types of molecules that can be used with the present invention. Nucleic acid microarrays allow a researcher to simultaneously view the level of thousands of different nucleic acid sequences present in a given sample. In addition to cellular process analysis, nucleic acid microarrays containing representative DNA sequences of many different viral genomes have been used for diagnostic purposes and were instrumental in the initial identification of the origin of the SARs virus. Human developmental studies have been dramatically altered as microarrays can analyze mRNA and protein expression changes in tissue over time. Clinical studies using microarrays to detect and predict differences in an individual's response to drugs also are being used to optimize chemotherapy treatment for some cancers.

The success of DNA, RNA, cDNA, aRNA, or oligonucleotide microarrays is based on the principle that identical strands of nucleic acids (sense and antisense strands) in solution will find each other and bind together or hybridize. The stringency of the hybridization reaction can be carefully controlled by those well versed in the art so that only exact, complementary nucleic acid sequences will hybridize. Microarrays use this ability of one nucleic acid sequence to bind to its exact complementary partner by fixing a spot containing copies of a known sequence (probe) to a known location on a stationary substrate and then applying an unknown sample containing fluorescently labeled nucleic acid sequences (target) to the substrate. If a complementary target sequence is present in the sample it will hybridize to a specific stationary probe spot on the array. After rinsing away any unbound target sample each spot is read by a scanner that today operates in one of two fundamental ways; confocal laser scanning or CCD image capture. With confocal laser scanning each probe spot 110 on the array is scanned in small sections or scan squares 100 between 2.5 and 10 micrometers in diameter resulting in dozens of individual brightness readings for an average probe spot 110 diameter of 80 micrometers, as shown in FIG. 1. In CCD imaging the array is illuminated with light of a specified or filtered wavelength and a specially configured CCD chip captures an image of the entire chip at one time. In both scanning methods a computer and specialized software is then used to calculate relative brightness levels between spots of interest and background light or reference spots and successful hybridization is estimated from these results.

Solid support microarrays, to which this patent applies, can be divided into three types depending on how the probe nucleic acids are affixed to the substrate. Mechanical spotting, as the name implies, uses pins or capillary tubes to apply a probe spot to the substrate. Piezoelectric techniques (ink jet printing) are used to spot nucleic acid building blocks or entire sequences to a substrate. Photolithographic techniques also are used to build nucleic acid probes at known locations on a silicon substrate.

Mechanical spotting gives a researcher large flexibility in designing experiments, is relatively simple and inexpensive, and most microarrays today are produced in this fashion. Ink jet synthesis of probe spots onto a solid substrate results in uniform, small spot size but the equipment is complex. This probe spotting technique has not gained wide acceptance in the marketplace. The high concentration of probe spots achieved with the later photolithographic technique,.manufactured and promoted by Affymetrix, Inc., has several advantages. The largest advantage is the ability to synthesize tens of thousands of probe spots onto a single substrate allowing the researcher to test for a broader range of substances on one array. Affymetrix is the largest commercial manufacturer of these synthesized arrays.

The production and successful use of a typical mechanically applied or piezoelectric (ink-jet) applied expression Microarrays involves a variety of equipment and chemical protocols including:
1) glass or plastic solid substrates coated to enhance the binding of spotted probes;
2) preparation of the nucleic acid probes;
3) apparatus for spotting the probes onto the substrate;
4) preparation of the fluorescently tagged or biotin labeled target nucleic acid samples;
5) hot plates, ovens, UV cross-linkers, hybridization chambers, rockers, water baths; and rinsing stations to affix the nucleic acid probes to the array surface, to hybridize the target sample to the array, and to apply if needed anti-biotin fluorescent tags to the array; and
6) scanner and software to excite the fluorescent tags at hybridization sites and interpret the emitted light intensity levels.

Affymetrix has produced equipment to simplify the processing of its synthesized (photolithographic) expression arrays comprising a method which includes using the apparatus in the following steps:
1) Affymetrix silicon array is pre-spotted with probe sequences of interest;
2) preparation of biotin labeled target nucleic acid samples;
3) Affymetrix fluidics and hybridization instruments for hybridization of the target sample to the array, application of anti-biotin primary antibody conjugate, and application of secondary antibody-fluorescent tag conjugate; and
4) scanner and software to excite the fluorescent tags at hybridization sites and interpret the emitted light intensity levels.

The dense synthesized arrays of Affymetrix not only can test for the largest number of substances, but, by offering standardized array substrates that combine steps 1) thru 3) of the mechanical or ink-jet generated arrays above, variability in experimental results is greatly reduced. A major drawback, however, of the Affymetrix microarrays and processing equipment, including the scanner, is there very high cost. The cost of scanner hardware and software, in the range of $40,000 to $65,000, represents one of the three most expensive pieces of equipment needed to use microarrays.

Mechanically spotted arrays offer low cost, flexibility, and control making this microarray technique the most widely used. However, there is no common glass substrate and no uniform treatments for probe attachment. There are also multiple fluorescent labeling molecules available and many versions of scanner instrumentation for the reading of these arrays. Together with other processing steps, the variability in substrates, probe fixation techniques, fluorescent molecules and their attachment, and scanning methods, contribute to large variances between individual mechanically spotted array results. The same variables apply to ink-jet generated arrays. The variances are enough to make comparisons between two different arrays extremely difficult and nearly impossible between two different research centers.

The scanning of fluorescent tags is common to all of the above microarray techniques. In addition to the expense of today's scanners, the instruments are sensitive and not easily portable. Some of the problems that the current light based scanner systems encounter are summarized below.
1) Background noise resulting from organic molecules fluorescing when struck by laser light. Contamination of the array surface with any organic material causes background light to be emitted that interferes with the desired light signal. Extensive software algorithms are used to subtract out this background noise but variance in background from array to array or between laboratories remains a key concern to users of arrays.
2) Confocal lenses are used in most of today's scanners to limit the depth of focus for the measured spot and thus reduce the amount of background light received by the detector. This narrow plane of focus limits the effectiveness of the scanner in gathering all relevant light signals from a true hybridization event.
3) The operator must set the energy level of the applied excitation laser. If the energy level is set too high, the fluorescent tags will bleach out and subsequent scanning gives only very reduced signals. To get reliable readings, the normally required energy bleaches most fluorescent signals so that they can be read reliably no more than three times.
4) Either the lens assembly itself or a mirror must mechanically move back and forth across the array to focus on each portion of a spot to analyze. This mechanical movement is inherently imprecise and limits the ruggedness of the instrument.
5) Present scanner systems convert an undefined level of electrical energy to a laser excitation signal. The light passes through a undefined space to the array, where the light is absorbed by the non-standardized fluorescent tags on the target molecules and converted to a longer wavelength of emitted light. The emitted light travels back through an undefined space and a filter. A portion of the light in a shallow plane (focus) is mechanically selected by a lens and/or mirror apparatus. The light is then converted to an electrical signal and stored for analysis. This lengthy, multi-step approach compounds variances in each step of the process of reading an array.

The other major imaging technique for arrays once again uses fluorescent tags on the target molecules. The entire microarray is bathed in light of a specific wavelength to excite a fluorescent tag and a CCD image sensor operating at extremely cold temperatures captures a full array image. Maintenance of this cold temperature requires expensive and cumbersome equipment. In addition, the various sources of light used, the undefined spacing to and from the array, the non-uniform fluorescent tags and the conversion of excitation light to emitted light energy, and the temperature sensitivity of a CCD sensor all combine to make this light sensing method also less than ideal.

A need therefore exists to standardize on a sensing substrate for mechanical and piezoelectric spotted arrays and a need exists to improve upon the fluorescent sensing of hybridization events used in all microarray platforms, including photolithographic arrays. Any technique that is developed to improve upon the confocal fluorescent or CCD scanners in use today must have the ability to take multiple, individual readings from a single probe spot. Following from this, any new technique must make thousands of independent measurements across an entire array to generate statistically significant predictions of hybridization. A new scanning system must be able to detect low levels of hybridization while being inexpensive, robust, small, repeatable, and intuitively easy to use compared to light based scanner detection. Significant improvements in these areas will contribute to a more widespread adoption of microarray technology.

2. Description of the Related Art

A search of the prior art located the following United States patents which are believed to be representative of the present state of the prior art: U.S. Pat. No. 5,284,748, issued Feb. 8, 1994; U.S. Pat. No. 5,137,827, issued Aug. 11, 1992; U.S. Pat. No. 4,794,089, issued Dec. 27, 1988; U.S. patent Publication No. US 2003/0003523 A1, published Jan. 2, 2003; U.S. Pat. No. 6,333,200 B1, issued Dec. 25, 2001; U.S. Pat. No. 5,891,630, issued Apr. 6, 1999; U.S. Pat. No. 5,532,128, issued Jul. 2, 1996; U.S. Pat. No. 5,567,301, issued Oct. 22, 1996; U.S. Pat. No. 5,466,348, issued Nov. 14, 1995; U.S. Pat. No. 6,355,491, issued Mar. 12, 2002; and U.S. patent Publication No. US 2002/01648.19 A1, published Nov. 7, 2002.

The affinity of certain biogenic substances to bind to one another has long been used to purify substances away from unknown sample mixtures. Sense and antisense strands of nucleic acids display this binding affinity. Southern Blotting, developed by Ed Southern in 1975, used immobilized unknown DNA strands on a membrane and known labeled DNA strands in solution to identify the presence and location of specific DNA sequences. Today the most popular microarray methods use known nucleic acid sequences as the immobilized elements (probes) and the unknown, labeled nucleic acid sequences (targets) are present in a sample solution. Current practice favors solid substrates for microarray use rather than membranes as the nonporous nature of solid substrates allows smaller, immobilized, known probe spots to be placed on an array thus increasing the amount of information provided by each array experiment. The known nucleotide sequences (probes) can be spotted or synthesized onto the sense chip using mechanical, piezoelectric (ink-jet), or photolithographic methods. The unknown sample nucleic acid sequences (targets) are either chemically labeled with a vitamin (biotin) or copies of the sample nucleotide sequences are synthesized incorporating fluorescently labeled nucleotides or biotin-labeled nucleotides. The labeled sample is then applied to the array and hybridization of complementary stationary probe and labeled target sequences is allowed to occur. Any unbound sample is washed away and, if the target was labeled with fluorescent label, the array is then read by a laser scanner. If the target was labeled with biotin then a streptavidin-fluorescent molecule conjugate, streptavidin being well known for its affinity to biotin, is applied and unbound conjugate is rinsed away. The array is then laser scanned and light emitted from the fluorescent tags is detected and quantified by the scanner.

Many different types of apparatus and methods have been described to produce a device that can electrically detect biomolecules, including nucleic acids, proteins, and antigens and antibodies. Prior art tends to focus on the construction of detection sense sites. These may be generally grouped into resistive, capacitive, or inductive sense sites.

An example of resistive sensing techniques may be found in Mroczkowski, et al., U.S. Pat. No. 4,794,089 issued Dec. 27, 1988, U.S. Pat. No. 5,137,827 issued Aug. 11, 1992, and U.S. Pat. No. 5,567,301 issued Oct. 12, 1996, all of which are incorporated herein by reference. U.S. Pat. No. 5,567,301 describes a sense site consisting of two conductive pads placed on an essentially nonconductive substrate and separated by a gap of extremely small size. The pads are connected via traces to an ohmmeter that measures the DC resistance across the gap. A known antigen is poured into a specific gap and the edges of the associated conductive pads.

Preparation of the target molecules begins when groups of small conductive particles are each labeled with a single, unique antibody and added to an unknown sample solution. If an antigen specific to the antibody is present in the unknown sample it will bind to the antibody coating on a specific conductive particle. The free antigen takes up space on the antibody-particle and thus inhibits it from binding to an identical stationary probe (antigen) coating on a sense site. After application of the target sample to the substrate, hybridization takes place. The binding of appropriate stationary antigen probes to antibody-particle complexes brings the conductive particle into the sense site gap. Subsequent silver enhancement treatment coats the bound particles with a layer of conductive silver and will reduce the resistance across the sense site. A detailed description of the silver enhancement process may be found in Hayat, M. A., Ed., Immunogold-Silver Staining: Principles, Methods, and Applications, CRC Press. Boca Raton, Fla., 1995. If free-floating antigen was present in the unknown sample, it will bind to the antibody particle and the conductivity of the sense site will be low. If no free-floating antigen was present in the unknown sample, conductivity at a given sense site will be high.

Various substances are described as suitable substrate materials for this resistive sense site, including glass and plastic, while various metal oxides, including chromium oxide, and other materials are presented as bioreactive substances that attract biological molecules to their surface. The different substrate materials and different bioreactive layers have different inherent resistance values and changing them can lower or raise the sense site resistance. The Mroczkowski, et al. patents suggest changing the bioreactive layers to lower the sense site gap resistance which allows more current to flow and improves the ability to see small changes in conductance (sensitivity) of the sense site to partial bridging of silver enhanced particles in the gap. However, changing to a different bioreactive layer material or a different substrate material in order to vary the base resistance also dramatically changes the binding affinity of all sense sites on the sense chip. This will make comparison between different sense chips difficult if not impossible. An improved method of varying sense site resistance, while keeping the bioreactive layer and the substrate material unchanged, would allow more comparable results between sense chips of different sensitivity (base current levels). The sense site of the present invention accomplishes this.

Additionally, a layout configuration of multiple, individually addressable, sense sites on one substrate is given. Upon examination, this layout will not work unless antigen-antibody binding occurs only infrequently and at sense-sites that are well separated. If several neighboring sense sites are conductive (the gaps closed), parasitic parallel paths to ground are produced which will increase the conducted current and distort accurate resistive measurements. This parasitic problem can not be overcome with external circuitry. The problem becomes severe when several hundred, or thousand, purposely-conductive sense sites are found in close proximity on an array. As a result, the layout described in the Mroczkowski patents does not work for detecting large numbers of closely spaced conductive sense sites.

Lastly, the Mroczkowski, et al. patents do not define how to isolate the first of a pair of binding reagents into microscopically small sense sites.

Jensen, Pat. No. US 2003/0003523 A1 issued Jan. 2, 2003, builds upon the above Mroczkowski, et al., patents by expanding the binding reaction components to include nucleic acid sequences (probes) affixed to the gap and the unknown solution containing biotin labeled nucleic acids serving as targets. Jensen describes a streptavidin-horseradish peroxidase (HRP) conjugate that can be bound to the biotin label of the hybridized targets. This HRP enzyme reduces metal ions in solution as it oxidizes a substrate and the reduced metal precipitates out of solution contributing to the conductive path across the gap. The precipitated metal is further enhanced with metal treatments before detecting or reading the sense site. Using the resistive gap sense site of the Mroczkowski, et al., patents, Jensen teaches that resistance at hybridized conductive sense sites drops more using the HRP conjugate than the conductive particle-silver enhancement treatment of Mroczkowski, et al. No methods are described that solve the problems of using the Mroczkowski patents to measure a large number of closely spaced conductive sense sites, to vary gap resistance, or to isolate the first of a pair of binding reagents in microscopically small sense sites.

U.S. Pat. No. 6,333,200 B1 issued Dec. 25, 2001, describes a resistive measurement technique similar again to the Mroczkowski, et al., patents. Successful hybridization is detected by bridging a small gap between two conductors photolithographically laid down on glass. The U.S. Pat. No. 6,333,200 patent provides a solution for getting probe substances located solely in the sense site gaps. The probe molecules first are affixed to latex coated magnetic particles. The particles are then attracted to bind in the gap of a particular sense site by applying an alternating voltage to individual pairs of conductive traces. The resulting dielectrophoretic force accumulates the probe-coated magnetic beads into the sense site gap. Applying an AC signal to a matrix of sense sites is extremely difficult and, once again, the shortcomings of the Mroczkowski resistive approach are not addressed.

Capacitive measurement using photolithographically defined electrodes to develop a biosensor are described in Newman Et al., W.D. Proc. Int. Meet. Chem. Sens., 2^{nd,} 1986, 6-23, 5966-598. A form of capacitive sensing is found in U.S. Pat. No. 5,532,128 issued Jul. 12., 1996, and U.S. Pat. No. 5,466,348 issued Nov. 14, 1995, to Eggers, et al. Semiconductor manufacturing techniques are used to construct a well in an insulation layer and a conductive plate of bioreactive metal is affixed to the bottom of the well. Probe molecules are affixed to the surface of the plate in the bottom of the well. A conductive ring is then placed around the periphery of the entire sense chip and serves as the second plate of a capacitor for this well, and all other wells on the chip. Alternatively, the two plates of the capacitor are constructed on the walls of an individual well. The probe substance is applied and affixed to the interior of the well and or onto the surface of the two plates. The subsequent hybridization of charged target molecules, such as nucleic acid sequences, to the probe changes a frequency dependent characteristic of capacitance between the plates. Similar to the purposes of the present invention, Eggers, et al., teach these capacitive sense sites as being:produced using semiconductor processing techniques resulting in extremely small sense sites and “millions” of sense sites on a single chip. Eggers, et al., specify the use of bioreactive metals and metal oxides, similar to the Mroczkowski et al., patents to attract and capture probe molecules to sense sites. In addition, Eggers, et al., describe functionalizing reagents to treat the sense site well interior to make it receptive to capturing probe molecules. Neither a process nor a method to apply and affix these reagents solely to the sense site well and not to the entire surface of the array are disclosed by Eggers, et al. Also, the ability to produce vertical metal coatings (capacitor plates) on only two sides of an etched well is not a trivial challenge for semiconductor processing. At a minimum this represents an expensive manufacturing technique. Additionally, a difficulty with this or any capacitive sensing technique is the need apply an AC signal to record a measurement. The need to apply AC signals of varying frequency to individual sense sites results in complicated on chip addressing and switching circuitry not specified by Eggers, et al. In addition, the air gap between electrodes in the open-air-capacitor elements of the Eggers, et al., patents adds to the variability of sense site capacitance results from electrode pitch tolerance, residual probe or target material, humidity, and temperature changes as compared to resistive sense site elements. These shortcomings prevent this approach from being used to construct dense reliable sensing arrays.

An additional capacitive measurement technique is described in U.S. Pat. No. 5,567,301, issued Oct. 22, 1996. Each sense chip is comprised of one large sense site consisting of discrete, stand-alone islands of metal placed on a non-conductive substrate. Two electrodes are placed upon the substrate and islands of metal at a considerable distance apart, 1.3 mm. The probe substance is affixed to the non-conductive substrate available between the metal islands. Target material is hybridized to the stationary probes resulting in a change in the resistive and capacitive components of this sense surface's AC impedance. This technique requires a relatively large sense site area and does not lend itself well to miniaturization. It also presents the same challenges to construction of dense sense sites as confronted by capacitive sensing and is susceptible to deformation from mechanical spot deposition.

The hybridization of charged biogenic substances to a top gate of a field effect capacitor or to the gate elements of various types of field effect transistors can affect the electrical characteristics of these devices in a measurable way and are described in U.S. Pat. No. 5,466,348, issued Nov. 14, 1995. Difficulties in energizing individual sense sites and the extreme complexity of the semiconductor elements described make this approach difficult to implement cost effectively. In addition, residual charge remaining in the depletion or enhancement channel of these devices may influence precise readings and should be removed, which leads to additional complexity and challenges the feasibility of this approach.

Electrical sensing techniques using inductance or magnetism to sort reactants, attract reactants to specific locations, or sense hybridization have been defined. Patent Publication No. US 2002/0164819 A1, published Nov. 7, 2002, shows an inductive site shaped as a bucket that attracts magnetic particles coated with biomolecules. In addition, the sense site can be energized to attract a large magnetic bead added to the target solution that will cover or act as a lid on the sense site bucket. Lastly the presence of magnetic particles at the sense site changes the inductance of the element. This publication teaches that the method of applying an external magnetic field and sensing current changes as magnetic particles pass through the sensor coils can be used to quantify material entering or leaving the sense site. The inductance of the site is determined by the application of an AC signal to the sense site/inductor. Both of these techniques require bi-directional access to each sense site. As with the capacitive techniques described earlier, this requirement makes simple, dense construction of sense sites extremely complex and practically difficult. The connection layout to sense sites prescribed in this patent publication is line intensive and does not lend itself to dense sense site construction. By contrast, FIGS. 13 and 14 from U.S. Pat. No. 6,355,491 B1, issued Mar. 12, 2002, to Zhou, et al., defines an inductive element produced in silicon that is used to attract (or repel) biomolecules from individual locations thus speeding up the reactions that might otherwise be dependent on slower passive diffusion.

Hybridization in this patent is not detected via conductance but by traditional fluorescent tagging of target molecules and light detection techniques and, therefore, does not apply to a discussion of electrical sensing. The complex nature of individually-addressing inductive sites, however, is presented and is useful for illustrative purposes.

### DETAILED DESCRIPTION OF RELATED ART

Constructing resistive sense sites is taught by Mroczkowski et al., U.S. Pat. No. 5,284,748 issued Feb. 8, 1994, and shown in FIGS. 5 and 6. In FIG. 5, a resistive layer that is bioreactive 45A (attracts and binds biomolecules) is laid down upon a non-conductive substrate 40 such as a glass slide. Conductive traces or leads 52 are placed upon the bioreactive layer and between these leads a probe substance 53 (antigen or antibody) is poured into place. A trough or depression is formed between the two conductive traces but specific well geometry is not specified. FIG. 6 defines another method of constructing a resistive sense site. Conductive leads 52 are laid down directly upon a non-conductive substrate 40, typically glass. Over this is applied a bioreactive layer 45B that contacts both the conductive leads and, similar to FIG. 5, forms a trough or depression between said leads. A probe material (antigen or antibody) is poured into the trough 53. Isolation of the bioreactive layer 45A from adjacent sense sites is not disclosed.

FIG. 7 shows the sense site layout and a proposed interconnect layout to attain multiple sense sites on one substrate from the Mroczkowski, et al., patents. The layout plan uses both sides of the substrate to obtain the greatest sense site density. FIG. 8 shows another embodiment of a multiple sense site layout taught by Mroczkowski that keeps all interconnect traces on one side or plane of the substrate and brings all connections to one edge of the substrate. This embodiment of interconnect is extremely trace line intensive and requires a separate connection to the edge of the board for every individual sense site. FIG. 9 represents the interconnect layout of FIG. 7 redrawn to illustrate how parasitic conductive paths can develop with multiple sense sites made conductive in close proximity to one another, thus causing failure of the measurement system.

Capacitive sense sites are shown in FIGS. 10 and 11 as taught by Eggers et al., in U.S. Pat. No. 5,891,630 issued Apr. 6, 1999. FIG. 10B shows a capacitor formed between a conductive plate 24a on the bottom of an individual well and a conductive ring located some distance from, and around the periphery of, the entire sense chip 15 and 24b. FIG. 11 shows another form of the sense site with a capacitor formed between two conductive plates 24a and 24b. In both drawings a well, formed in an insulating, layer such as silicon dioxide, is described in which probe and target hybridizations take place. Multiple sense sites can be replicated on a semiconductor substrate under this method. The production of a well with two perpendicular conductive plates and two sides bare as in FIG. 11 is not a trivial task in semiconductor processing and, if manufacturable, significantly increases production costs. The implementation of multiple sense sites becomes further complicated when the required interconnection circuitry, not shown, is added to accommodate AC measurement signals.

Further, application of probe molecules to the defined sense sites is practically limited to the use of bioreactive substances in the construction of the well components. Eggers, et al., in U.S. Pat. No. 5,891,630, specify that the capacitor plates could be produced from metals such as gold, platinum, and titanium, or various metal oxides. These metals can bind to organic thiol groups that have been incorporated into the probe molecules. This method and apparatus has the disadvantage of requiring the probe material to contain bound thiol groups. The use of metals or oxides to bind the probe molecules at a sense site is reminiscent of the methods used by Mroczkowski, et al., where different resistive polymers and metal oxides are used as a bioreactive layer to attract and bind probe molecules in the resistive sense site gap. This method should work. However, a more flexible way to use one sense chip platform to attract and bind your probe of interest is to condition (functionalize) the sense site gap substrate with a reagent so that optimal binding with a probe of interest will occur. Many such reagents are known in the art including, without limitation, amino-silane, epoxy silane, and poly-1-lysine. Each binds better with certain probe substances than others. Eggers, et al., in U.S. Pat. No. 5,891,630, disclose several of these substances but fail to specify how to apply the substances to the sense sites. The usual method of applying these substances does not work when an operator wants to apply them to only a microscopic spot and not to the adjacent area on the array. Currently the functionalizing reagent (sometimes called the bioreactive substance) is applied to the entire surface of the array in liquid form where it is allowed to react and covalently bind to the substrate. After rinsing away unbound reagent, probe spots are then applied to the surface. Heat or UV crosslinking are used to induce covalent bonding of probe to the bioreactive layer. Lastly, to insure that unbound areas of the bioreactive layer do not bind to sample target molecules, the entire array is bathed in a blocking reagent that binds to all free substrate/bioreactive areas that are not covered with probe.

As discussed above, the current methodologies for applying a bioreactive layer apply and affix the material over the surface of the entire sense chip. This alone could electrically link neighboring sense sites and influence capacitance readings. The only way to place bioreactive material into a single sense site then is to microscopically apply bioreactive liquid individually to each site. This is not feasible.

The construction of bioreactive metals or metal oxides as the conductive elements of a capacitor, or as the bioreactive layer between conductive elements in a resistive sense site, is a practical method to place the bioreactive layer in completely separate, microscopic sense site gaps on a microarray. With the placement of the bioractive layer addressed with this method, however, the problem of isolating probe reagent into only the sense sites and not the sense chip surface still remains. The apparatus and method of the present invention solves the problem of applying reagents (bioreactive, probe, or target) to independent and separate microscopic sense sites.

Inductive properties are disclosed in U.S. patent Publication No. US 2002/0164819 A1 to attract magnetic particles coated with biomolecules. Of note, the interconnect required for inductive measurement, like capacitive measurement, is line intensive because of the need to manipulate AC signals (FIG. 12). FIGS. 13.and 14 detail another proposed interconnect layout for an inductive sense site as taught by U.S. Pat. No. 6,355,491 B1. Each Row and each Column requires 2 bonding pads (FIG. 13) as well as on chip active elements (FIG. 14) to directionally control the current in and out of each sense site. The complexity of this sense site also makes production costly.

In summary, the prior art for biomolecular, electrical sense sites does not provide for efficient interconnection and efficient individual sense site addressing. The prior art does not define how to: efficiently produce, adjust or vary the sense site gap resistance; produce microscopic resistive sense sites that contain a minute amount of any of a variety of bioreactive substances used for probe attachment and separated from neighboring sense sites; produce microscopic sense sites that have a probe substance deposited in a multitude of neighboring sense sites from one probe spot deposition wherein each sense site and its corresponding probe material are completely separated and independent from neighboring sense sites; produce microscopic sense sites that are robust to electrical damage from operator and machine handling; produce microscopic sense sites that break the surface tension of applied liquids; or produce microscopic resistive sense sites that can withstand the mechanical stress of contact probe spotting as well as the chemical processing for probe synthesis.

Further, the prior art fails to define: how to control substrate temperature for resistive measurements; how to program the average current level or resistivity of the sense chip sites to meet a precise specification; a structure or method to test the sensor for full conductance during manufacturing; or a method to protect the sense site from excessive current or test instrument short circuits. The combination of these and other novel techniques disclosed in the present invention allow the production and use of sense chips patterned with the most space efficient matrix of sense sites, limited only by semiconductor production tolerances, and capable of improving on current light detection scanning techniques.

### BRIEF SUMMARY OF THE INVENTION

The present invention provides a means to electrically detect a binding reaction between two or more substances, particularly between nucleotides, or proteins and ligands, or antigens and antibodies. The invention improves upon the resistive sensor described in U.S. Pat. No. 5,284,748 to make a sensing system that can replace current microarray laser scanning techniques. The method of the invention involves bringing the two or more substances together so that the binding reaction between them causes full or partial closing of an essentially open electrical circuit. The resulting change in the electrical resistance or conductance of the circuit indicates a successful binding reaction.

Unlike the prior art, the present invention creates versions of a new sense site architecture that can be used with the prevalent microarray contact printing, ink-jet printing, or photolithographic sample deposition systems. The present invention is based on using semiconductor substrates and processing techniques to vary the electrical characteristics of the substrate and create a dense matrix of sense sites, each no more than 50 microns from its nearest neighbor, on a single, inexpensive, durable yet disposable, sense chip. Each sense site on the chip consists of a planar semiconductor diode (or other unidirectional device) connected to one of two conductive traces located on opposite sides of a sense-gap. The sense gap consists of two conductive traces each connected to, but separated by, a substrate material, or combination substrate material and bioreactive layer, that is considerably less conductive than the two traces. The gap substrate in the present invention is semiconductor material such as silicon or germanium that can be doped with either N or P material to vary the gap resistance as required. A bioreactive layer may be constructed on top of the substrate (such as chromium or chromium oxide), or a liquid, atomized or gaseous bioreactive substance may be applied to the sense sites after construction.

In the case of applying the bioreactive layer to the sense sites after sense chip construction, a novel method is presented in the present invention that results in a layer of bioreactive substance, such as amino-silane, affixed to the top of the microscopic gap substrate and the edges of the two conductive leads of the sense site. Various well known bioreactive substances can be used to affix a multitude of different probe molecules in the sense site gap. The same technique is also presented to isolate probe material and blocking reagents in only the sense site gaps.

In another embodiment of the present invention, the bioreactive layer is constructed into the sense site during production of the sense chip. The sense gap, in this instance, may be a thin layer of any of a number of bioreactive metals, metal oxides, plastics, or polymers on top of an N— or P-doped silicon substrate as depicted in FIGS. 42 and 43.

The surface of the chip, except for openings at the sense site gaps and bonding pads, is coated with a passivation layer that serves to make the top surface of the chip as planar as possible to withstand the stress of contact printing of samples. The combination of isolation layers and the passivation layer also produces wells over the sense sites. These wells, together with the bead mop method of the present invention as detailed herein, serve to separate the probe sample contained in a single spot into many independent sites for potential hybridization. This separation step, in turn, is a critical feature of the present invention which allows multiple readings of a single application of a probe spot to the array surface thus providing statistically meaningful analysis of hybridization events. A matrix of conductive rows and columns with sense sites interconnected is constructed so that each sense site can be individually addressed and read by on-chip or off-chip circuitry. The shape of the sense site leads is defined to improve the sense chips resistance to electrical damage from operator and machine handling. The inclusion of an island of inert material such as oxide in the sense site gap or at the edge of the well serves to the break surface tension of applied liquids.

On the chip level, a programmable set of resistive links is produced in the detection circuitry path that allows total resistivity of the detection circuit to be standardized to one specification and results in more precise and comparable array-to-array results. A temperature-sensing element(s) is incorporated into the substrate to assist in controlling sense site chip temperature and thereby improving reading precision. A test sense-site is defined to measure full conductivity levels, and an overcurrent protection circuit to safeguard the sense chip against excessive current damage is included on the chip.

Other features, advantages, and objects of the present invention will become apparent with reference to the following description and accompanying drawings.

### BRIEF DESCRIPTION OF DRAWINGS

These and-other objects, advantages and-novel features of the invention will be more readily appreciated from the following detailed description when read in conjunction with the following drawings, in which:

FIG. 1 represents confocal laser scan pattern and probe spot existing in the art.

FIG. 2 represents a representative sense site pattern and probe spot of the present invention.

FIG. 3 represents confocal laser scanner block diagram existing in the art in focus.

FIG. 4 represents confocal laser scanner block diagram existing in the art out of focus.

FIG. 5 represents resistive sense site bioreactive layer below sense leads existing in the art from U.S. Pat. No. 5,284,748, issued Feb. 8, 1994.

FIG. 6 represents resistive sense site bioreactive layer above sense leads existing in the art from U.S. Pat. No. 5,284,748, issued Feb. 8, 1994.

FIG. 7 represents resistive sense site two-sided interconnect existing in the art from U.S. Pat. No. 5,284,748, issued Feb. 8, 1994.

FIG. 8 represents resistive sense site one-sided interconnect existing in the art from U.S. Pat. No. 5,284,748, issued Feb. 8, 1994.

FIG. 9 presents parasitic conductive paths two-sided interconnect existing in the art.

FIGS. 10A and 10B present capacitive sense site outer ring existing in the art from U.S. Pat. No. 5,532,128, issued Jul. 2, 1996.

FIG. 11 presents capacitive sense site vertical plates existing in the art from U.S. Pat. No. 5,532,128, issued Jul. 2, 1996.

FIG. 12 presents inductive capping of reaction sites interconnect existing in the art from U.S. patent Publication No. US 2002/0164819 A1, published Nov. 7, 2002.

FIG. 13 presents inductive sense site interconnect existing in the art from U.S. Pat. No. 6,355,491 B1, issued Mar. 12, 2002.

FIG. 14 presents inductive sense site interconnect detail existing in the art from U.S. Pat. No. 6,355,491 B1, issued Mar. 12, 2002.

FIG. 15 presents a 4×4 array of sense sites of the present invention showing the path of current flow.

FIG. 16 presents an 8×7 array of sense sites of the present invention.

FIGS. 17A-17D present examples of resistive sense site gap doping depth and size in various embodiments of the present invention.

FIGS. 18A-18D present examples of sense site lead shapes for optimal electrostatic discharge protection in various embodiments of the present invention.

FIG. 19 presents a top view of a center-placed liquid shunt of the present invention.

FIG. 20 presents a side view of a center-placed liquid shunt of the present invention.

FIG. 21 presents a top view of a well-side liquid shunt of the present invention.

FIG. 22 presents a side view of a well-side liquid shunt of the present invention.

FIG. 23 presents an example sense site diode and sense gap diffusion pattern of the present invention.

FIG. 24 presents an example of a side view of a resistive sense site construction with silicon oxide at the sense site of the present invention.

FIG. 25 presents an example of a side view of a resistive sense site construction without silicon oxide at the sense site of the present invention.

FIG. 26 presents an example of the operation of a magnetic or metallic bead in cleaning sense chip surface-bead mop of the present invention.

FIG. 27 presents an example of bioreactive agent applied to the entire array surface of an embodiment of the present invention.

FIG. 28 presents an example of bioreactive agent from FIG. 27 remaining in sense site wells and removed from non-sense site surface after bead mop of an embodiment of the present invention.

FIG. 29 presents an example of sense chip packaging of the present invention.

FIG. 30 presents an example of temperature sensing diode and chip packaging of an embodiment of the present invention.

FIG. 31 presents an example of laser trimmable test sites for full range conductance measurements of an embodiment of the present invention.

FIG. 32 depicts a programmable fuse bank of an embodiment of the present invention used to adjust average sense site circuit resistance to specification.

FIG. 33 is a circuit diagram depicting an example of overcurrent protection to safeguard sense sites of the present invention.

FIG. 34 is a circuit diagram depicting an example of serial control of sense site addressing of the present invention.

FIG. 35 is a circuit diagram depicting an example of parallel control of sense site addressing of the present invention.

FIG. 36 depicts a probe spot applied to a sense chip of an embodiment of the present invention.

FIG. 37 depicts a probe spot and blocking solution applied to a sense chip of an embodiment of the present invention.

FIG. 38 depicts a probe spot and blocking solution applied to a sense chip of FIG. 37 after rinsing/bead mop of the array for an embodiment of the present invention.

FIG. 39 depicts a side view of a probe bound to sense site of the present invention.

FIG. 40 depicts a side view of a probe and target hybridization at a sense site of the present invention.

FIG. 41 depicts a simplified side view of biotin streptavidin-colloidal gold binding of the present invention.

FIG. 42 depicts a simplified side view of silver enhancement of hybridized sense site with complete bridging by colloidal gold capped by silver layer of the present invention.

FIG. 43 depicts a simplified side view of silver enhancement of partially hybridized sense site with partial bridging by colloidal gold capped by silver layer of the present invention.

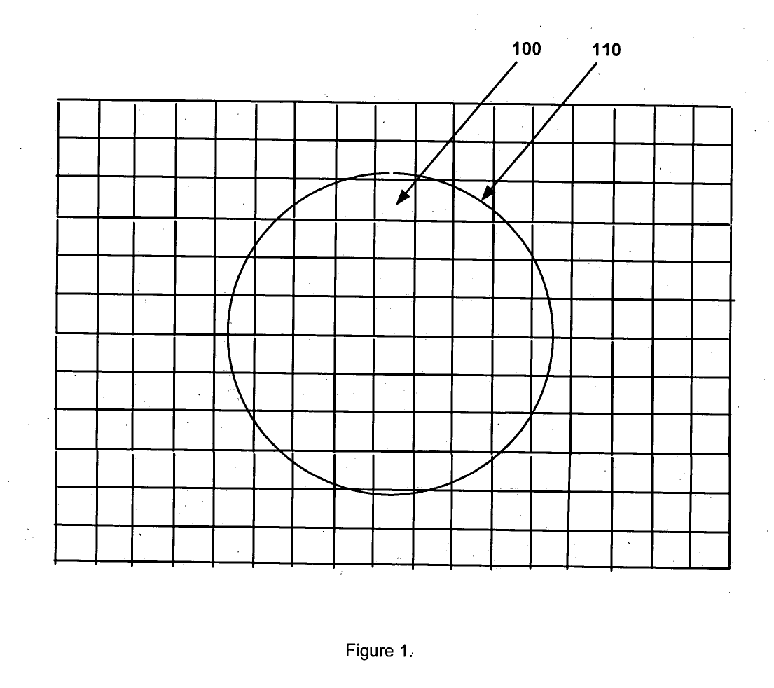

For simplicity in description, identical components are identified with the same numerals in this application.

### DETAILED DESCRIPTION OF THE INVENTION

The present invention is useful in the detection of specific nucleotide sequences, proteins, antigens, or antibodies. Any biomolecular substance, which has a binding affinity for and will hybridize to another biomolecule, can be detected using this novel apparatus and method.

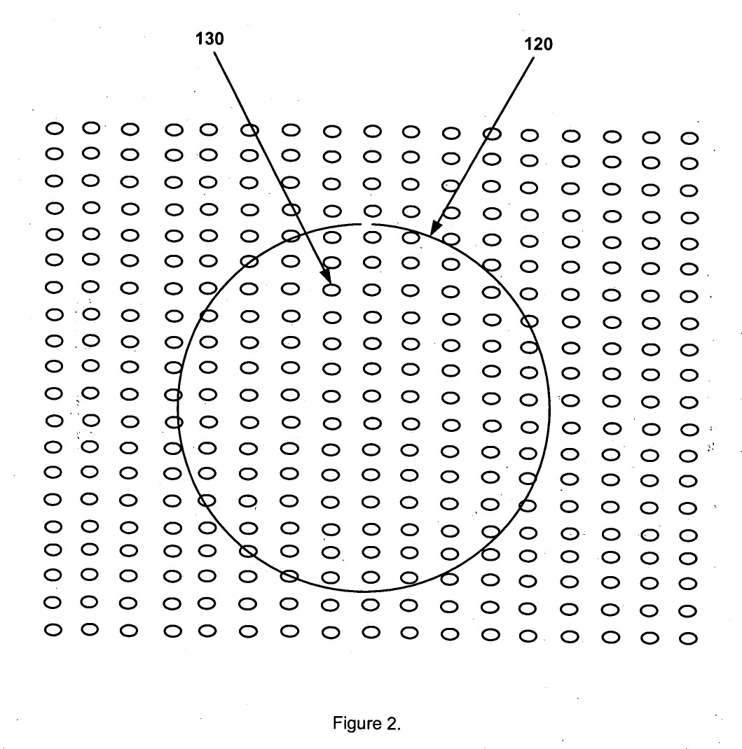

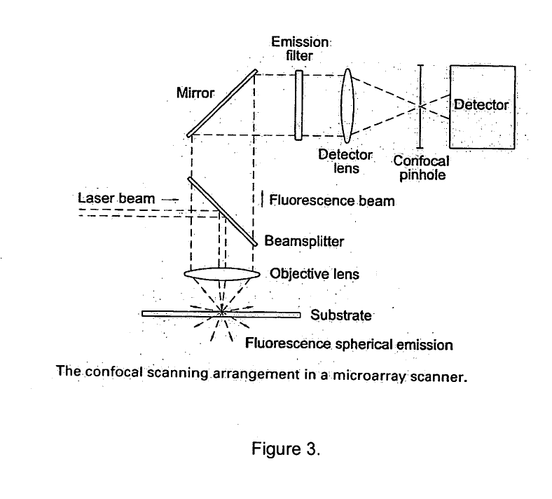

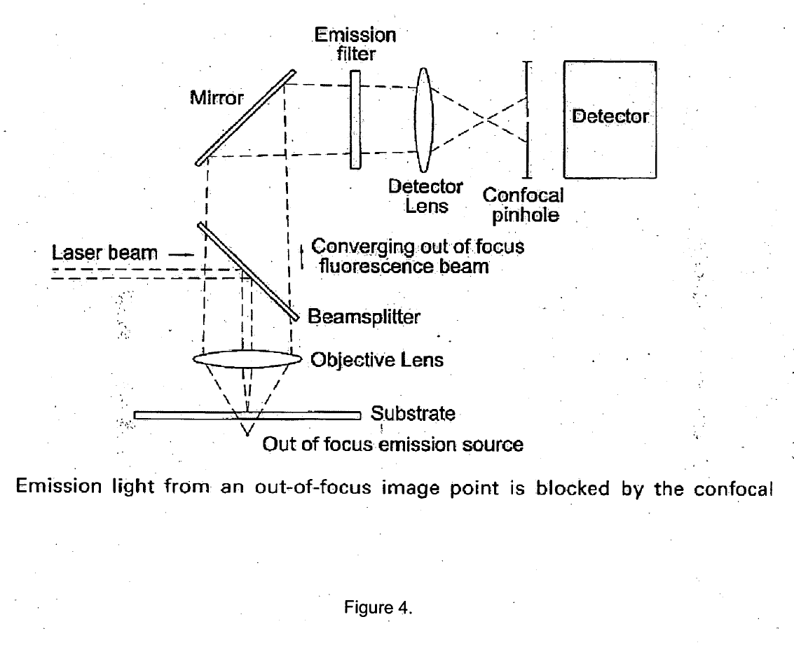

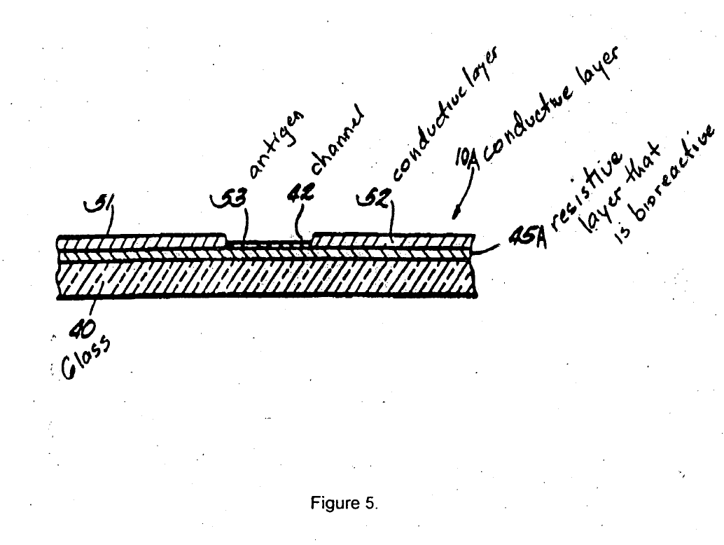

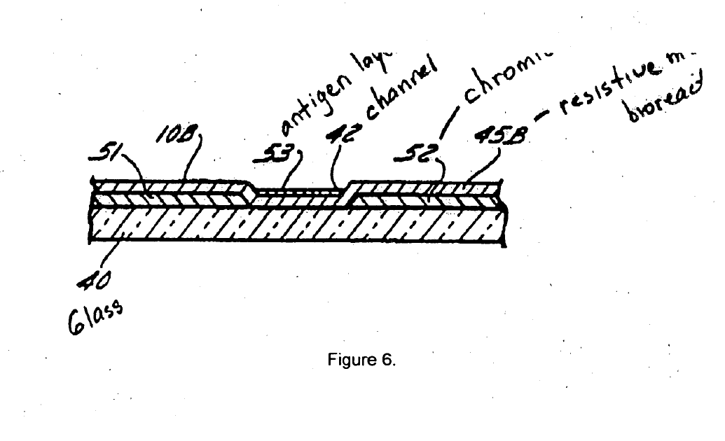

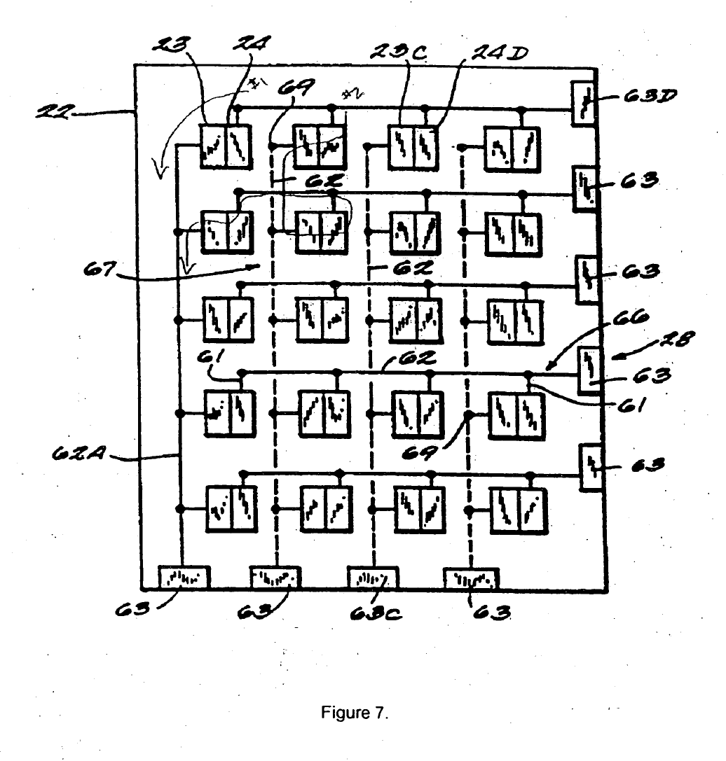

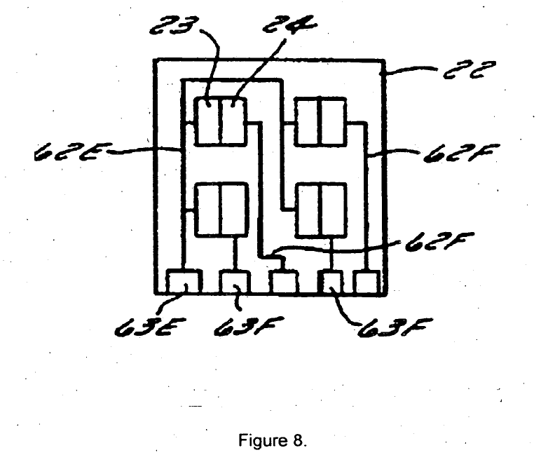

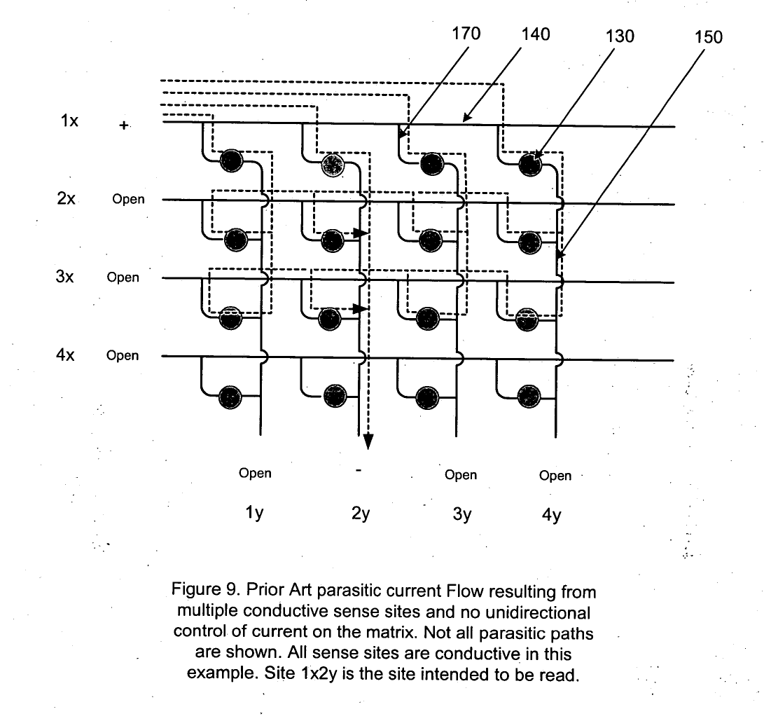

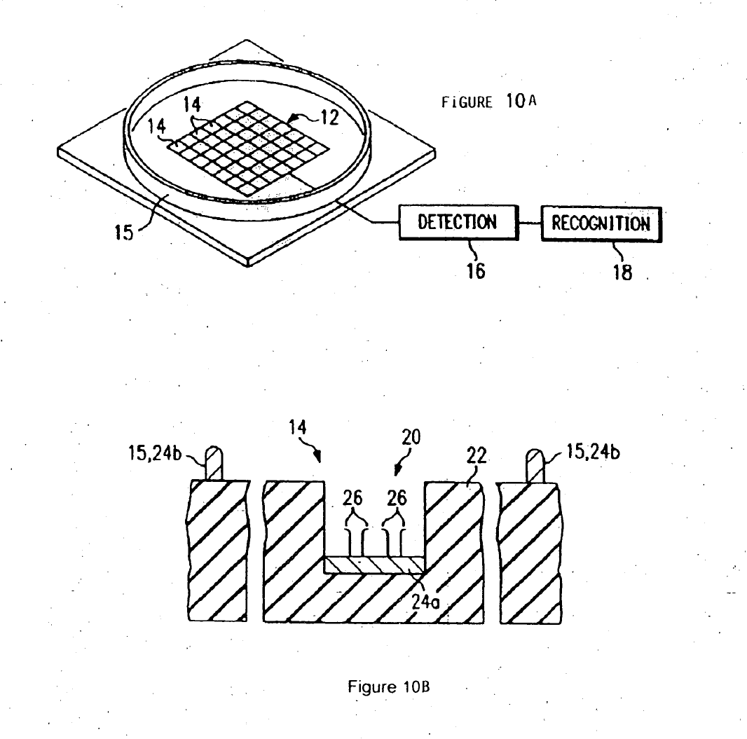

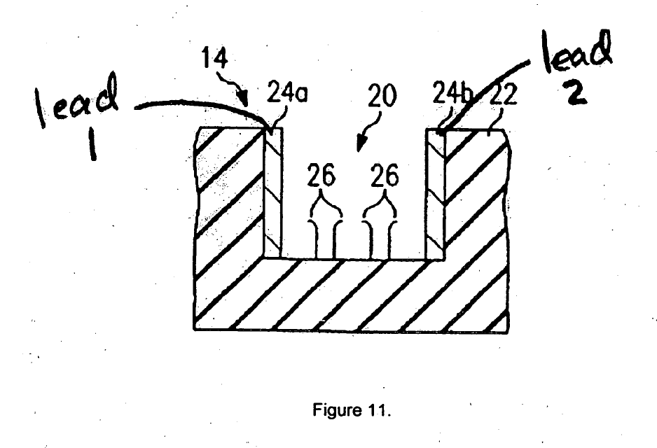

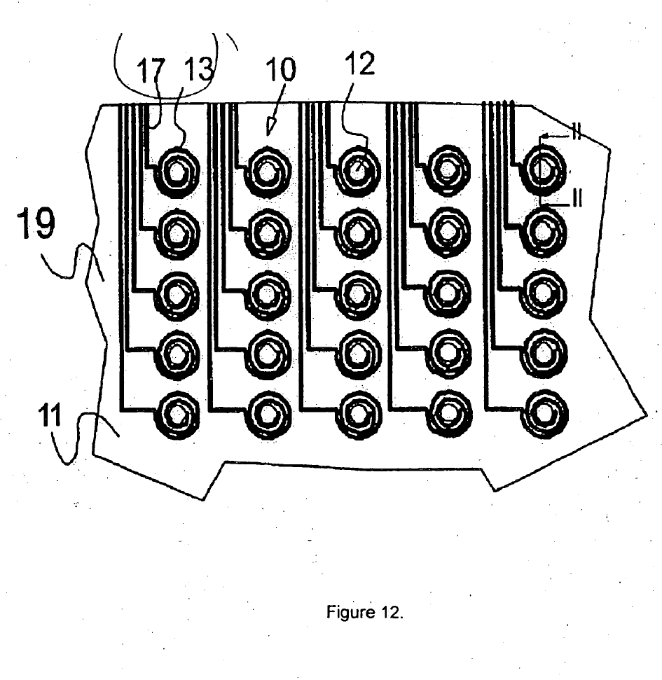

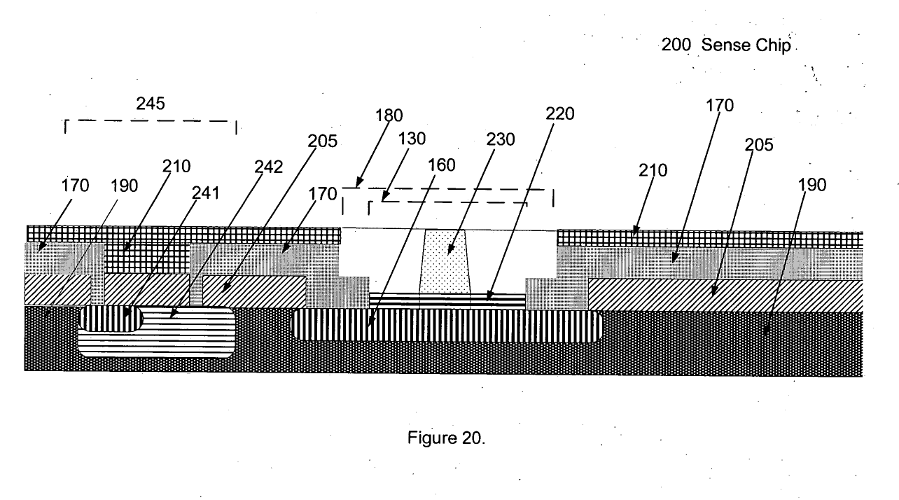

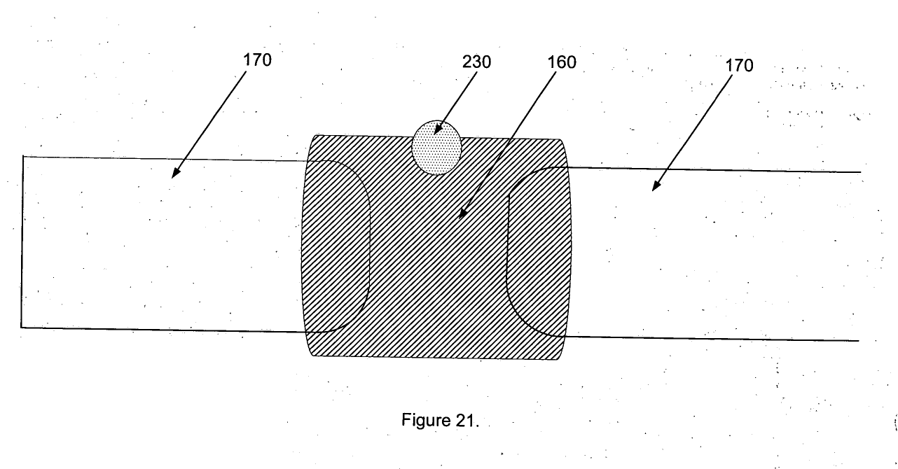

One part of the present invention is a new resistive sense site 130 to receive a probe DNA spot 120, as depicted in FIG. 2. As shown in FIG. 20, this novel sense site is produced using semiconductor substrate material 190 and comprises: (i) a planar semiconductor diode or unidirectional semiconductor device (for the sake of simplicity the diode element is not identified in the drawings as part of the sense site, for example FIG. 20, element 180; however, it is considered an integral and essential component of each sense site); (ii) a doped semiconductor region serving as the gap substrate 190; (iii) a pair of conductive traces with curved corners serving as sense site leads 170; (iv) a four sided well constructed from silicon oxide isolation layers 205 and a passivation layer 210; (v) a liquid shunt 230 of inert material standing perpendicular in the sense site gap 130 or protruding from the wall of the well; (vi) a bioreactive layer 220 coating the sense gap 130 and a portion of the sense site leads 170; and (vii) a passivation layer 210 serving to make the top surface of the sense chip level.

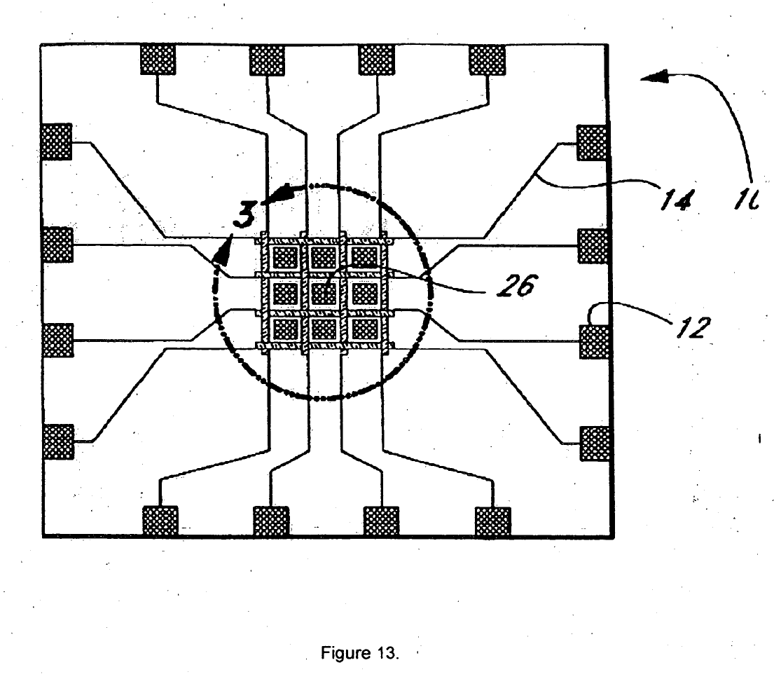

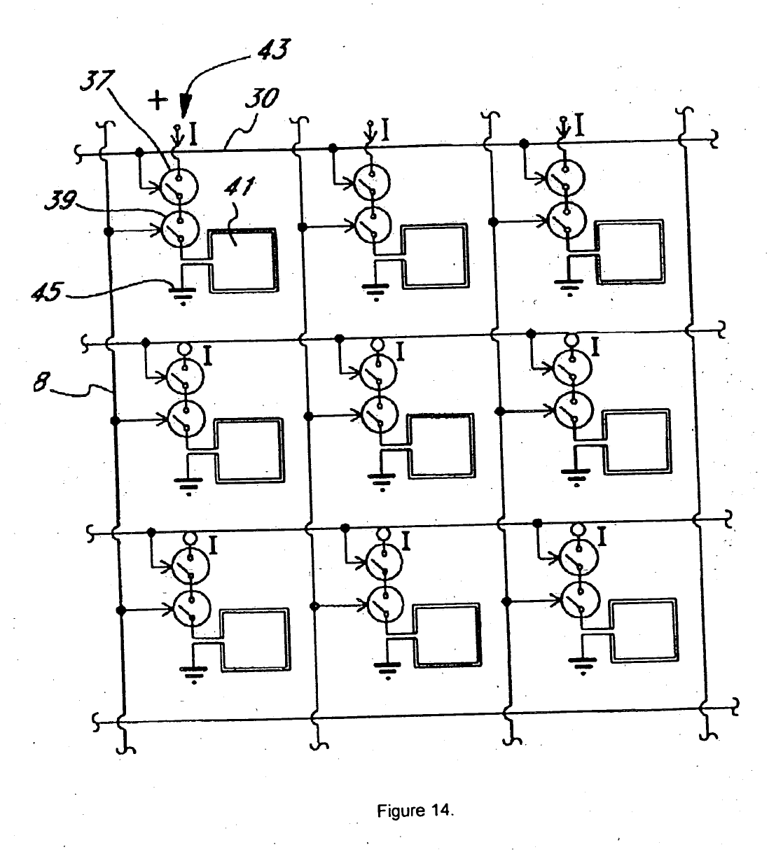

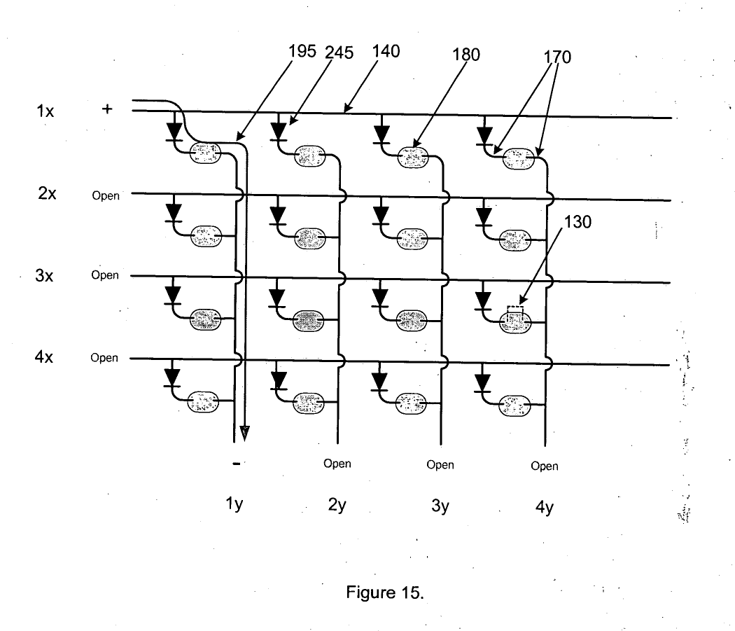

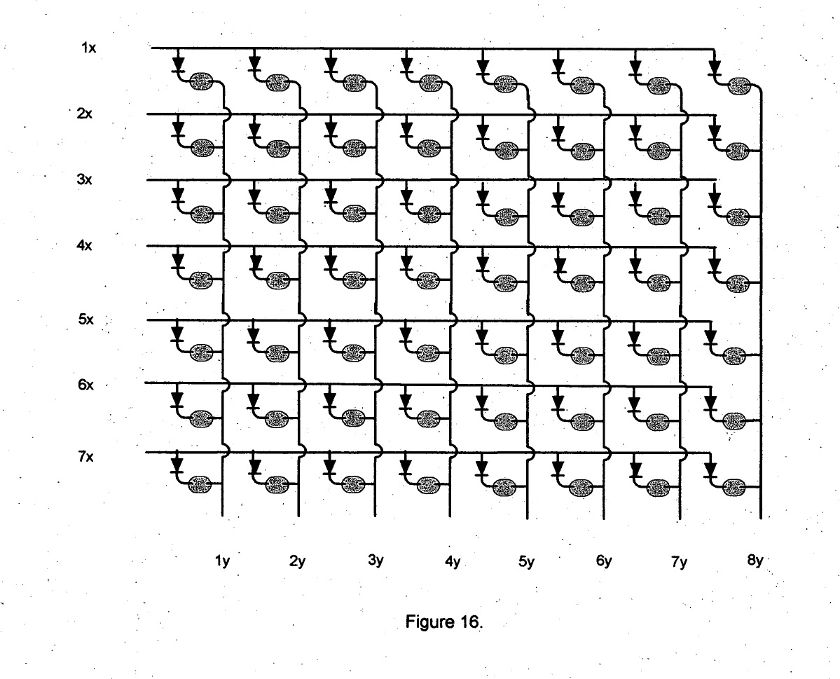

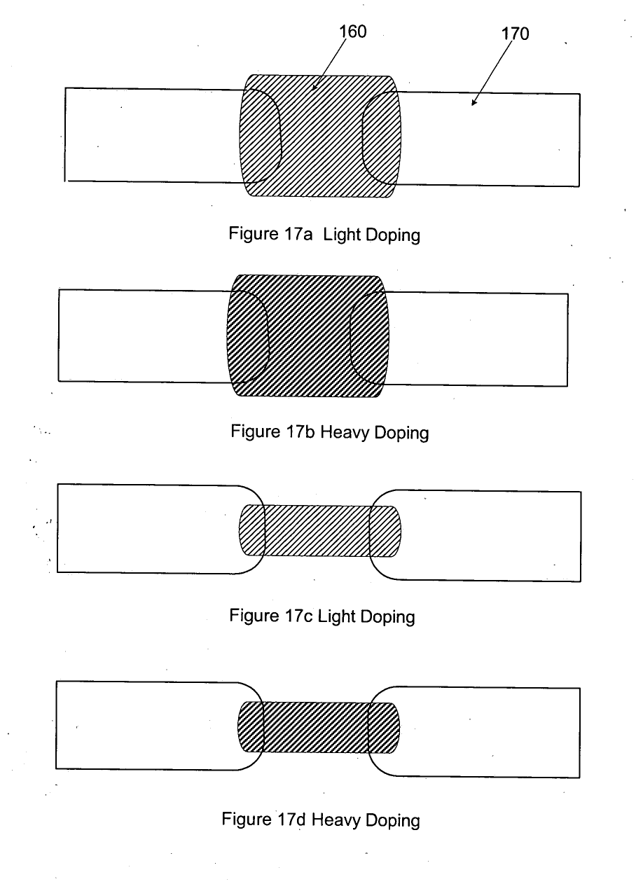

FIG. 15 is a representative top view of a sense site 180 array of the present invention comprising row interconnect 140 elements and column interconnect elements 150. A 4×4 array is shown with the conductive or current path 195 for measuring sense site (1x 1y). All sense sites are produced on a semiconductor substrate 190, representative by—but not limited to—silicon or germanium. Each sense site contains a unidirectional element such as a low leakage diode 245 in series with one of two conductive traces or sense leads 170 located on opposite sides of a sense-gap 130. The diode 245 should be produced so as to minimize leakage current. The present invention uses an X and Y matrix of interconnected rows 140 and columns 150 that energizes a considerable portion of the conductive sense site diodes in a reverse biased mode. By using low leakage devices 245, currents detrimental to the sensitivity of the described sense chip are minimized. The sense site 180 consists of two conductive traces connected to, and separated by, a substrate material and bioreactive layer that is more highly resistive than the two traces. The gap substrate in the present invention is semiconductor material that is doped with either N or P material to vary the gap resistance as needed. It is possible to have all sense sites 180 on a single sense chip have the same diffusion and same resistance, or it is possible to intermix different diffusion substances, diffusion depths, or the size of diffusion areas, at different sense sites to produce a sense chip with sense gaps of varying resistance. This can be useful in detecting a small amount of hybridization in a sense site gap 130 resulting in multiple, low resistivity hybridized areas connected in series across the gap (partial bridging). The lower the substrate resistance at an individual sense site, the more total current will flow and the easier it can be to detect small percentage variations in total resistance across the gap. However, making every sense site a wide, highly doped, low resistance, high current path will increase the operating power and reverse leakage currents of the sense chip. A balance of standard resistance and one or more levels of lower resistance sense sites on the same or separate sense chips allows the manufacturer to provide many different sense chips that strike varying balances between sensitivity, power consumption, and leakage currents. FIG. 16 shows an expanded top view of an array of the proposed resistive sense sites. FIGS. 17A-D show examples of the variation in sense gap diffusion 160 resulting from variations in doping density and doping area. This will result in different resistances between the sense leads 170 in the site gap.

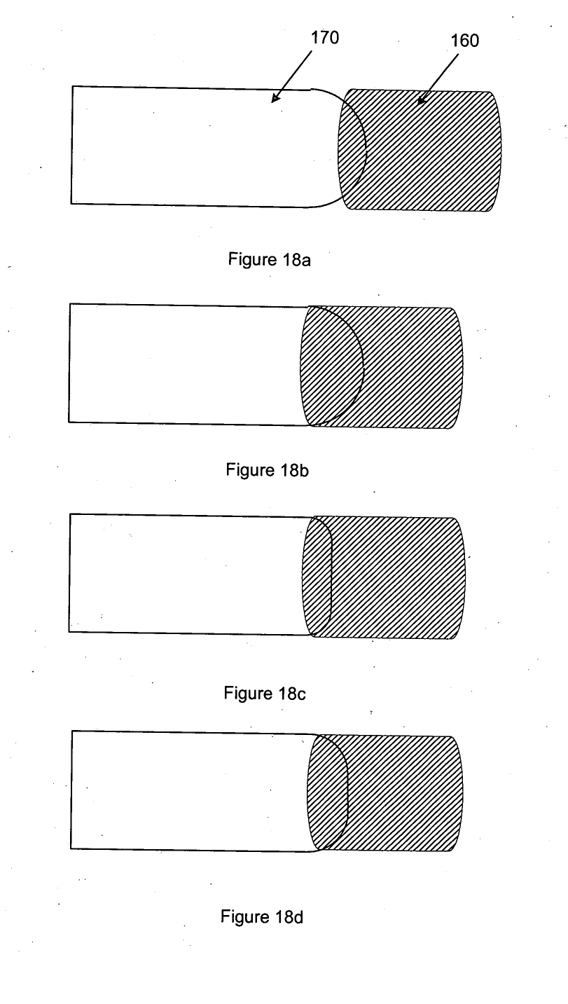

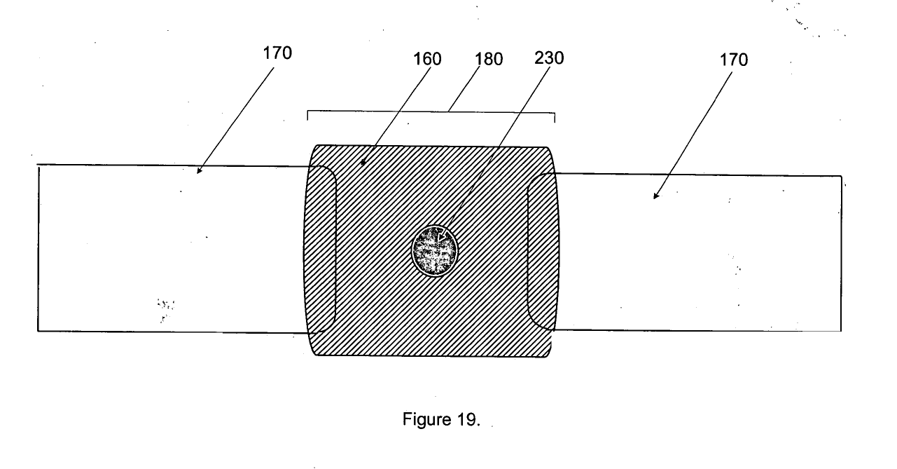

The envisioned sense chip of the present invention with a multitude of microscopic resistive sense sites will be handled by operators and come into contact with various instruments and appliances during a typical application of probe spots and subsequent fixation, rinsing, sample application, rinsing, conjugate application, rinsing, silver enhancement, rinsing, and electrical testing. The surface of the array is open to the environment and to possible mechanical and electrostatic damage to a greater degree than the usually completely encapsulated semiconductor device. Of particular note, handling precautions should be specified for the device. Steps should also be taken, however, to reduce potential damage to the sense chip from electrostatic discharge. The sense site leads should be configured so that they contain no exposed sharp edges. By rounding the sense site lead edges, electrical charge is prevented from accumulating at an angular point on the lead and the chance of excess charge aggregating and discharging to the nearby sense lead is thereby reduced. Thus, rounded leads significantly improve the reliability and robustness of the proposed array. FIGS. 18A-D show examples of rounded edges on the conductive sense leads 170 within the sense gap diffusion 160 which improve the chip's resistance to electrostatic discharge.

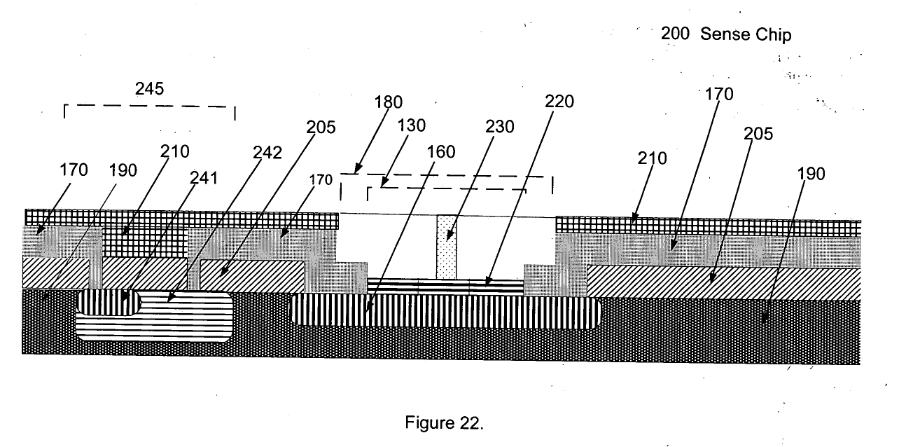

FIGS. 19-22 show possible embodiments of liquid shunts 230 for the present invention. These shunts 230, or protrusions, help to break the surface tension of applied liquids and allow the freer flow of liquid reagents in and out of the sense site gap 130. As the present invention is meant to be a testing platform or chip suitable for a variety of analyses, the viscosity of the various reagents may vary considerably from one application to the next. Often, applied liquids resist entering microscopic sense site wells 180 or gaps 130 with walls which are uniformly round, square, or rectangular in shape. Embodiments of the present invention envision including either stand alone protrusions or shunts 230, FIGS. 19 and 20, or protrusions that are incorporated into the side wall of the well or sense site 180, FIGS. 21 and 22, both of which are referred to as liquid shunts 230. Semiconductor processing techniques well known in the art can readily produce these features.

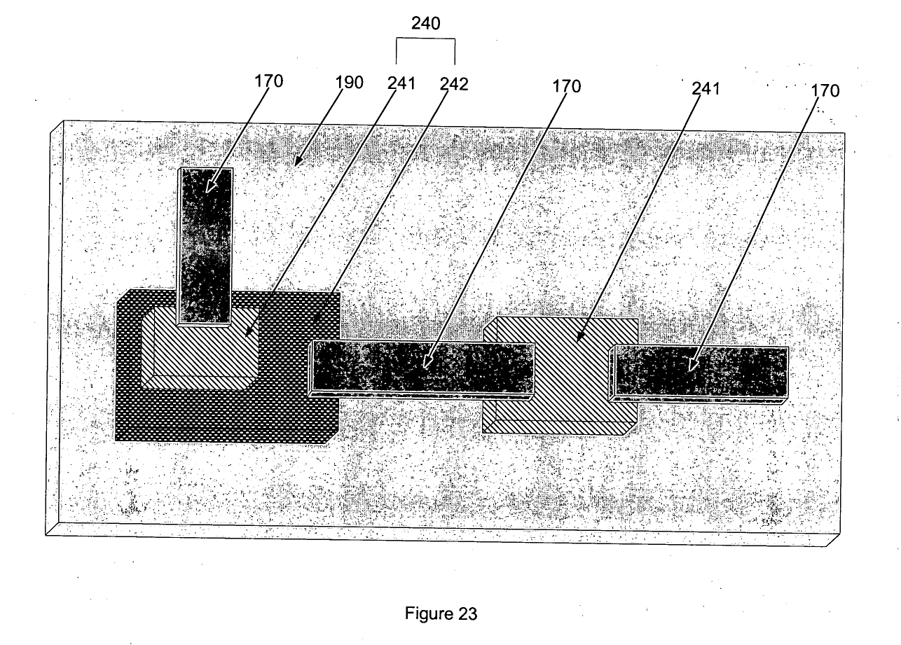

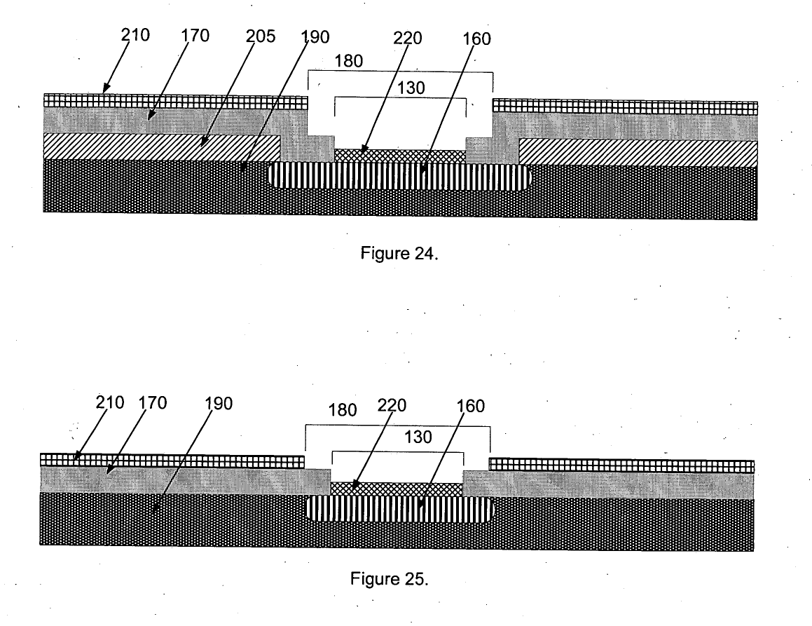

The ability to produce the prescribed sense chip 200 is based upon the resulting creation of a well around the sense site as the sense site is constructed, as depicted in FIGS. 23 and 24. FIG. 23 shows a semiconductor substrate 190 that has photolithographically selected areas diffused with reagents well known in the art, such as Argon and Boron, to modify the resistivity of the selected areas. 130 represents the gap area of the sense site and 240 represents the diffusions making up the planar diode of a typical sense site. Interconnects 140 or 150 connect the diode and sense site leads 170 and sense site 180 complex to a matrix of X and Y conductive rows 140 and columns 150. FIG. 24 depicts a side view of a resistive sense site 180 and sense gap 130. The isolation layer of silicon oxide 205, conductive traces or sense leads 170 and the final passivation layer 210 combine to create a four-sided depression that surrounds the semiconductor substrate gap. The passivation layer can be from a variety of substances known in the art and serves to improve the mechanical strength of the surface of the chip to the application of mechanical probe spots. The construction of all of the above elements is known in the art and presents no special challenges in manufacturing. If the sense chip is manufactured without a bioreactive layer, the surface of the semiconductor substrate at the bottom of the sense site well (the sense site gap 130), is open to the environment and is either prevented from coming into contact with oxygen during processing or any oxide coating is removed as one of the last steps in processing. If the sense gap substrate is doped silicon open to the environment, an effort will be made to shield the substrate and prevent oxidation. After additional processing outlined below, the chip is ready for application of a bioreactive material 225 to be applied and covalently bound to the exposed sense site gap substrate and sense site leads. FIG. 25 depicts an alternative structure of the sense site 130 that does not use a silicon oxide layer. Silicon oxide is used elsewhere on the chip but not near the sense site gap 130. By eliminating this oxide layer around the sense site gap 130, or by varying the thickness of the oxide if it is used, the height of the resulting well around the sense gap can be adjusted. If the height of the well is low, as shown in FIG. 25, the need for a liquid shunt may be eliminated.

It should be noted that the sense gap wells also provide a haven to shield the reactive areas of the sense chip from inadvertent contact. Accidental smudging and smearing of probe/target spots is reduced as a natural result of this architecture.

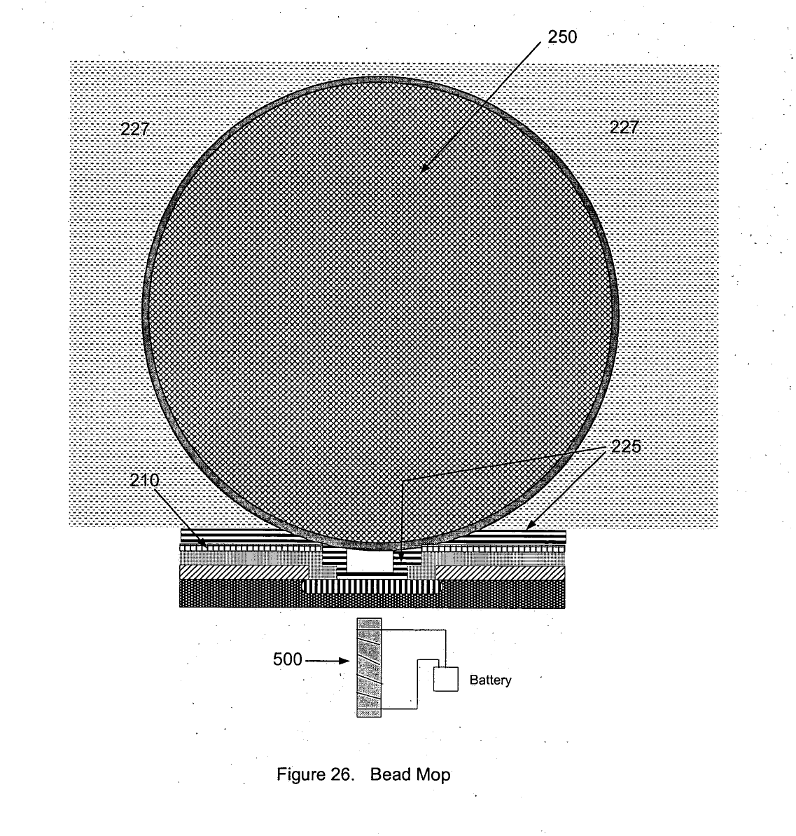

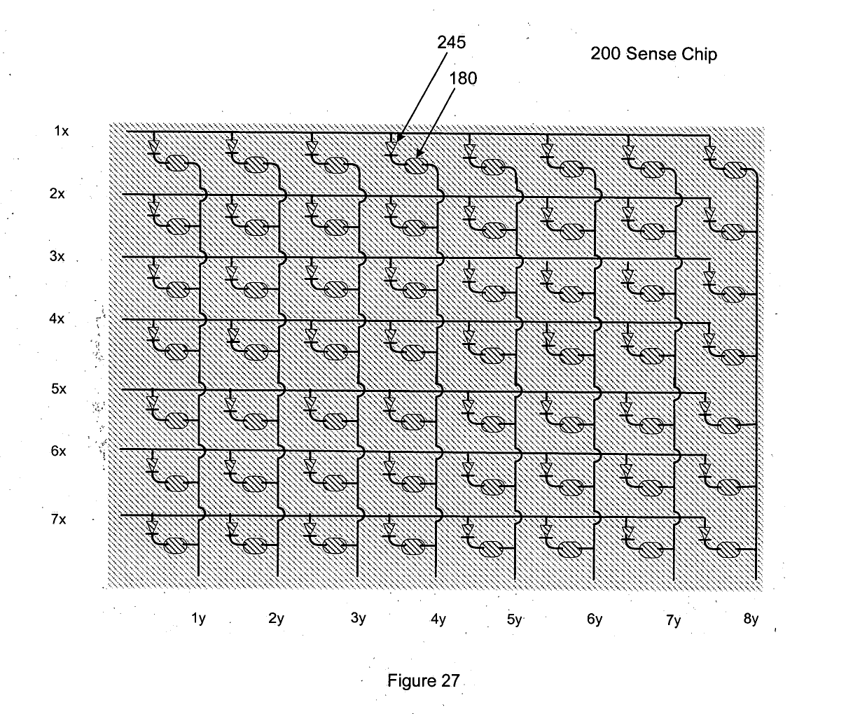

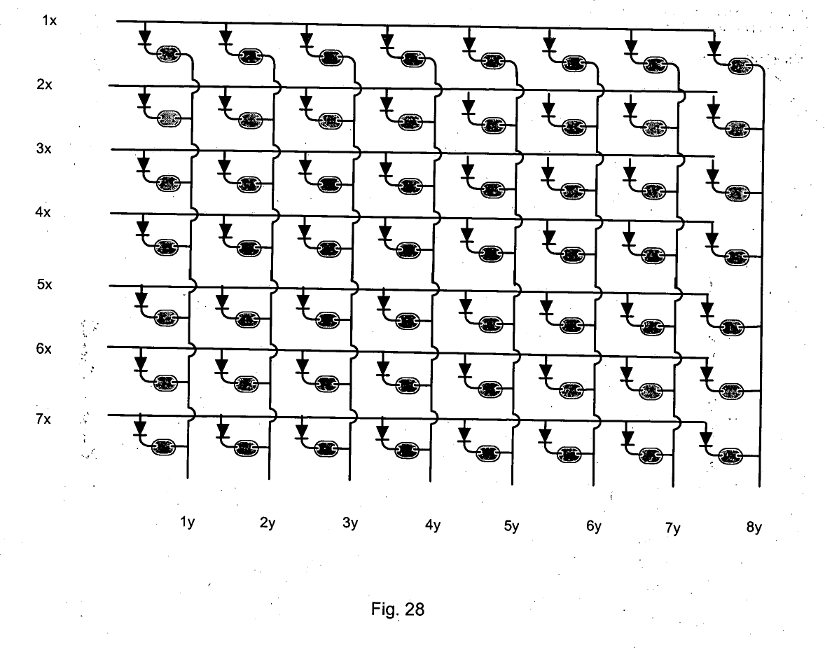

The construction method and materials described above result in a well surrounding the sense gap substrate. As described earlier, depositing a bioreactive substance or probe substance only in the microscopic well and not on the surrounding surface of the sense chip is a challenge. The present invention utilizes a metallic or magnetic bead 250, as shown in FIG. 26, coated with latex, or latex and a layer of substance which binds strongly to both the latex and the bioreactive reagent 225 that is on the sense chip surface, or no latex but a layer of substance that binds directly to the magnetic or metallic bead and bioreactive reagent on the surface of the chip or simply a charged bead of any material or a plain metallic or magnetic bead. Such coated and uncoated beads 250 are readily available from commercial manufacturers. The diameter of the beads should be selected so that they are sufficiently large so that they cannot enter or drop down into the wells created by the manufacture of the sense sites. The beads are added to an inert solution and the solution 227 is then placed over the surface of the sense chip 200, as depicted in FIG. 27, covering a plurality of sense sites 180 and associated diodes 245. Permanent magnet(s) and/or electromagnet(s) 500 positioned beneath the sense chip substrate attract the beads 250 from solution down to the surface of the sense chip, as depicted in FIG. 26. Movement of the substrate 190, external permanent magnets, or electromagnets, or varying an external magnetic field by randomly or sequentially energizing single or multiple electromagnets underneath or around the substrate will result in physically moving and rolling the beads around the surface or passivation layer 210 of the sense chip 200. As a result, the surface of the beads 250 will contact, bind, and remove or physically break away the bioreactive reagent 225 present on the surface or passivation layer 210 of the chip 200 but not the material present in the sense gap 130 depressions or wells. After an appropriate period of time, the mopping of the sense chip surface with the beads is complete and the magnetic field is removed. The solution is then rinsed away. This leaves a sense chip with a scrubbed passivation layer 215, and bioreactive layer 220 substance remaining only in all the sense site 180 wells and coating the exposed sense leads 170 and sense site gap substrates 190, as depicted in FIGS. 26 and 28. Probe molecules with an affinity for the bioreactive layer 220 will be drawn to and bind to the surface of the sense site wells. This bead mop method, FIG. 26, results in an unreacted, bioreactive coating of the researcher's choice, residing solely in the sense site wells, ready for further processing. It is envisioned that this step of bioreactive coating would be performed by the manufacturer but could be performed on completely blank sense chips by the user.

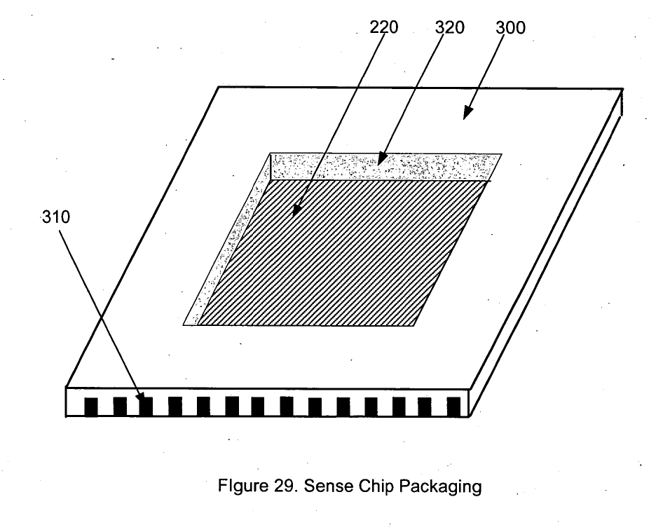

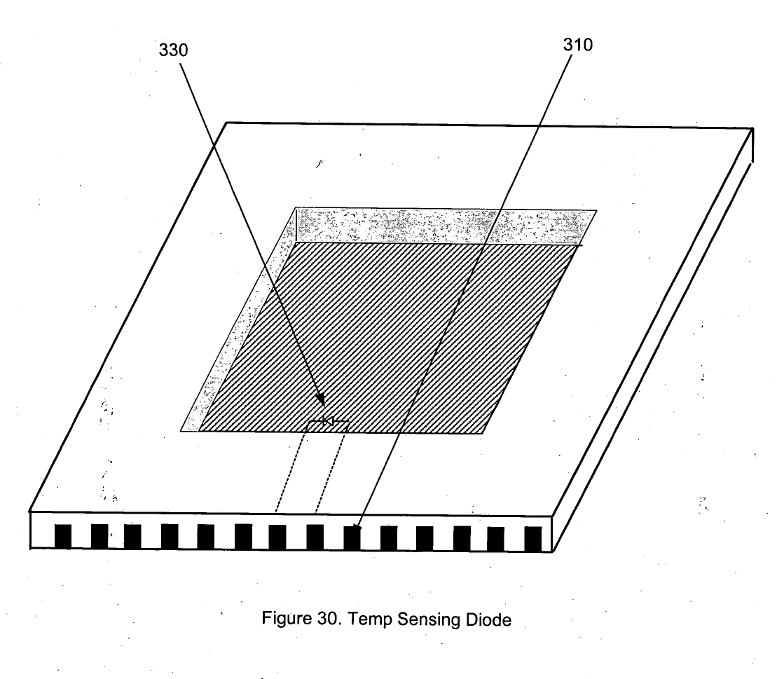

Following bioreactive coating of the sense chip, continuity, DC parametric, and functionality tests are performed. Units are laser trimmed if necessary to bring average conductance within specification and test sites are trimmed to open them at the conclusion of testing. Units that pass are packaged into a non-reactive package 300, as depicted in FIG. 29, comprising external pins 310 which serve as connections to the sense chip, and a package well 320. The package provides added mechanical strength and a platform to handle the sense chip. The package also brings appropriate electrical connections from the chip to a surface of the package and leaves the dense reactive matrix of the sense chip surface accessible for bioreactive reagent 220 addition and processing.

In addition to the sense sites described above, four additional hardware elements are included in the sense chip that greatly improve the precision and speed of measurements as well as the reliability, of the device. FIG. 30 shows the incorporation of a temperature sensitive element (diode) 310 that is connected to external contacts on the chip 310. Variations in the diode's reverse bias leakage current are characterized versus temperature and can be used to determine the chip temperature. The resistance of most materials is known to change with variations in temperature and thus controlling the sense chip temperature allows more precise, repeatable, and comparable readings to be made. To limit temperature variations an external heating and cooling element can adjust the sense chip temperature based on diode current fluctuations.

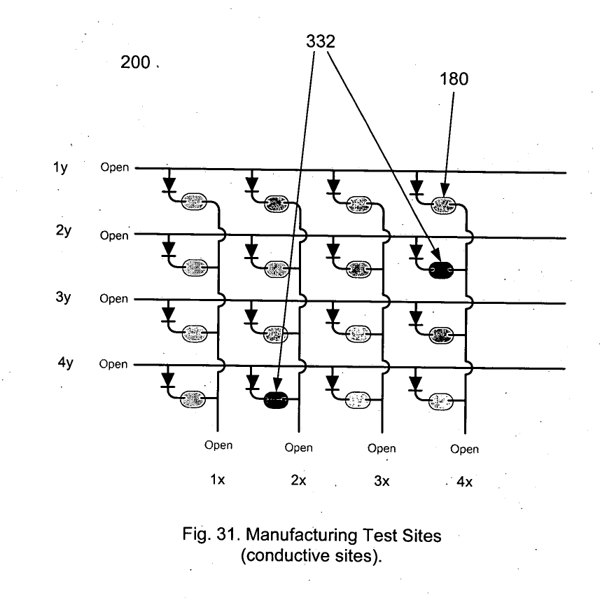

FIG. 31 represents a representative sense site 180 array and selected conductive test sites 332 with the sense gap bridged by a metal connection of known resistance. Several of these test sites 332 are located at dispersed sites on the chip 200 and allow the calibration of operating parameters prior to packaging of the sense chip. This allows production of more uniform and precise devices. The test sites are laser trimmed to open the sense gaps after manufacturing testing is complete. This preserves the integrity of the reversed biased sense site diode network that allows individual resistive sense site interrogation.

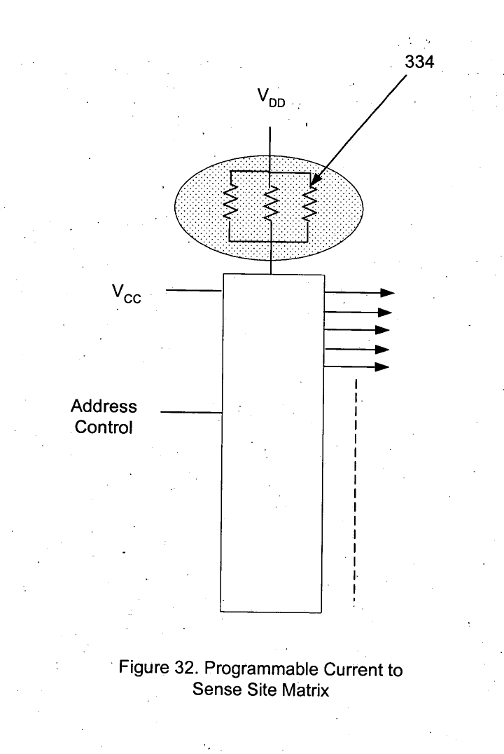

FIG. 32 shows a fuse link circuit. Laser trimming techniques applied at the time of wafer level test, and familiar to those versed in the art, can adjust the resistance 334 of this circuit. By trimming out resistive paths of this circuit, the average current through a sense site is adjusted to a tighter tolerance and results in improved chip-to-chip comparisons of test results.

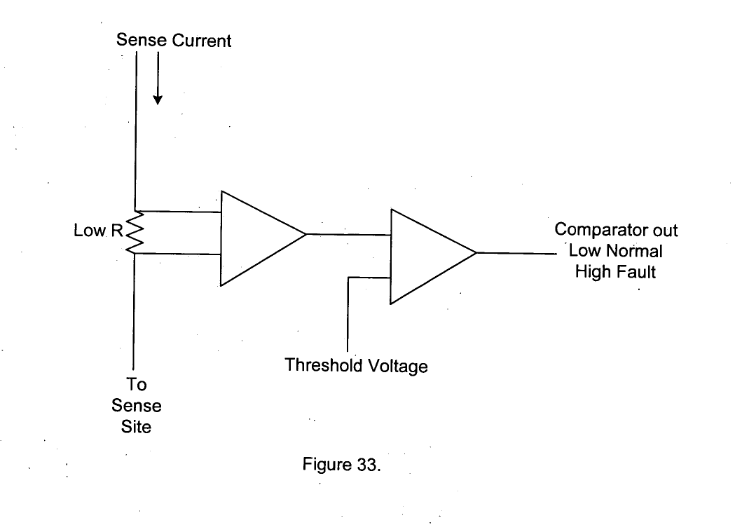

FIG. 33 shows an on-chip overcurrent protection circuit that will disconnect the sense chip from potentially damaging currents above a user defined threshold. This feature significantly improves the reliability of the electrical detection system and safeguards the valuable time and energy invested by the operator in processing the microarray.

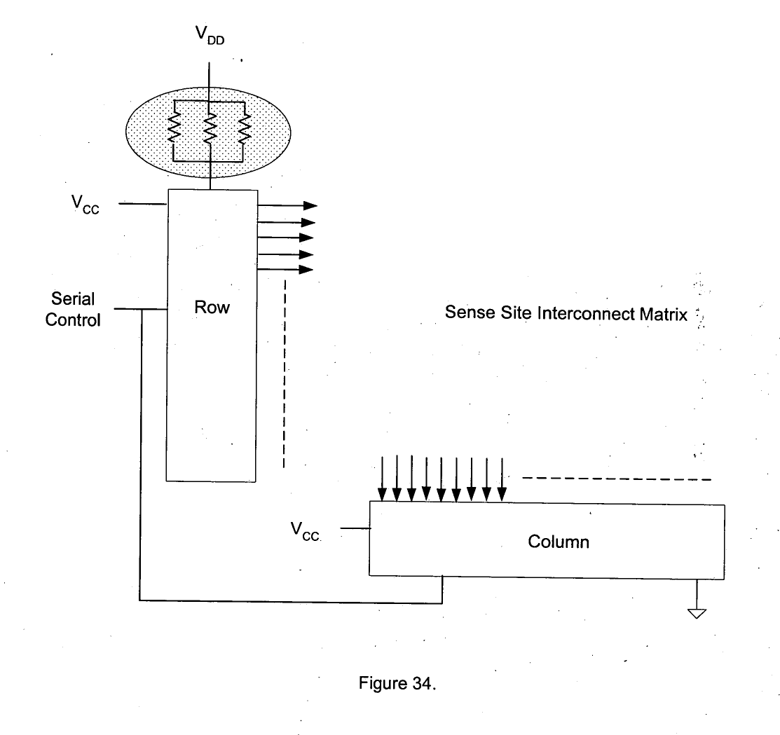

FIG. 34 shows a block diagram of serial control and FIG. 35 a parallel control version of the sense chip for the addressing of individual sense sites. Either method may be used as the serial method, while slower, greatly reduces the pin count of the sense chip and the parallel method, while pin intensive, speeds up the access to and the reading of chip.

It is anticipated that the ability to build useful electrical circuits in the same semiconductor substrate as the sense site matrix will be developed. Therefore, in addition to circuitry to interface the sense chip to a test instrument and on-chip row and column address circuitry, additional circuit functions and layout techniques may be employed to: (i) improve signal to noise ratios; (ii) stabilize on chip voltage and current levels; (iii) separate analog signal ground from logic device ground; (iv) amplify or attenuate signals; (v) compare and contrast signals; (vi) digitize and reconstruct signals; (vii) store and retrieve readings; and (viii) compare signals to values stored in memory.

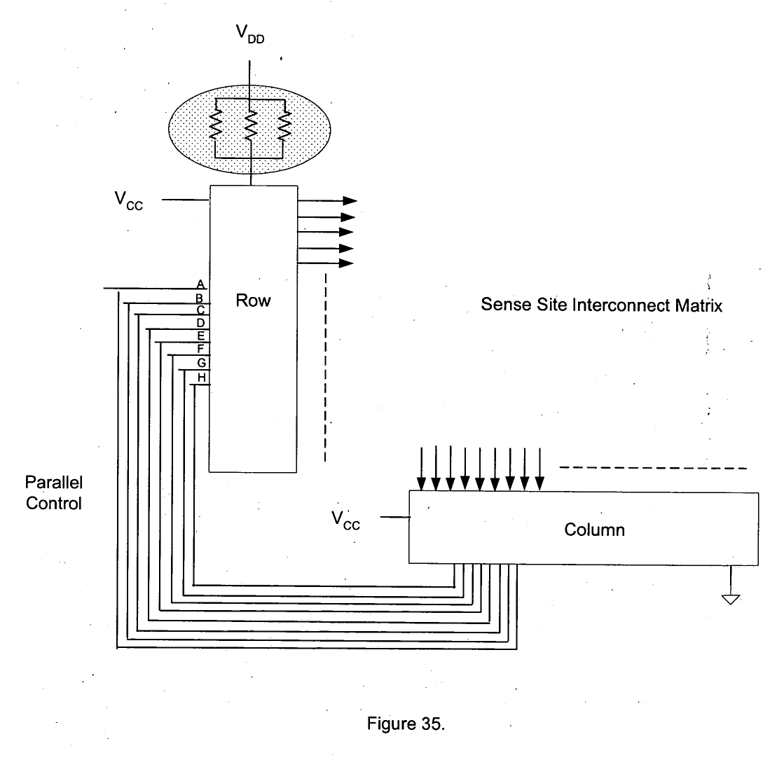

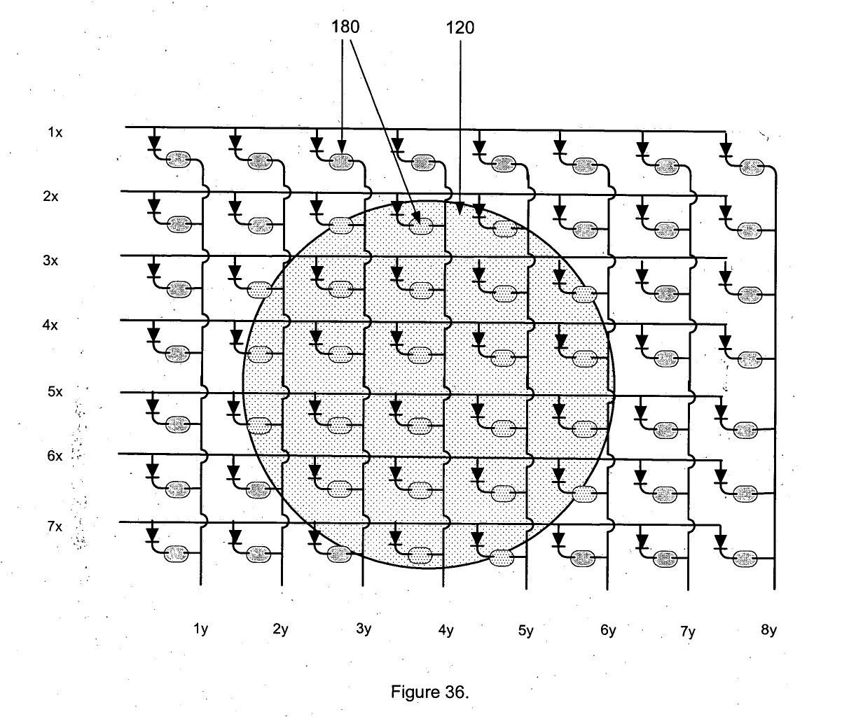

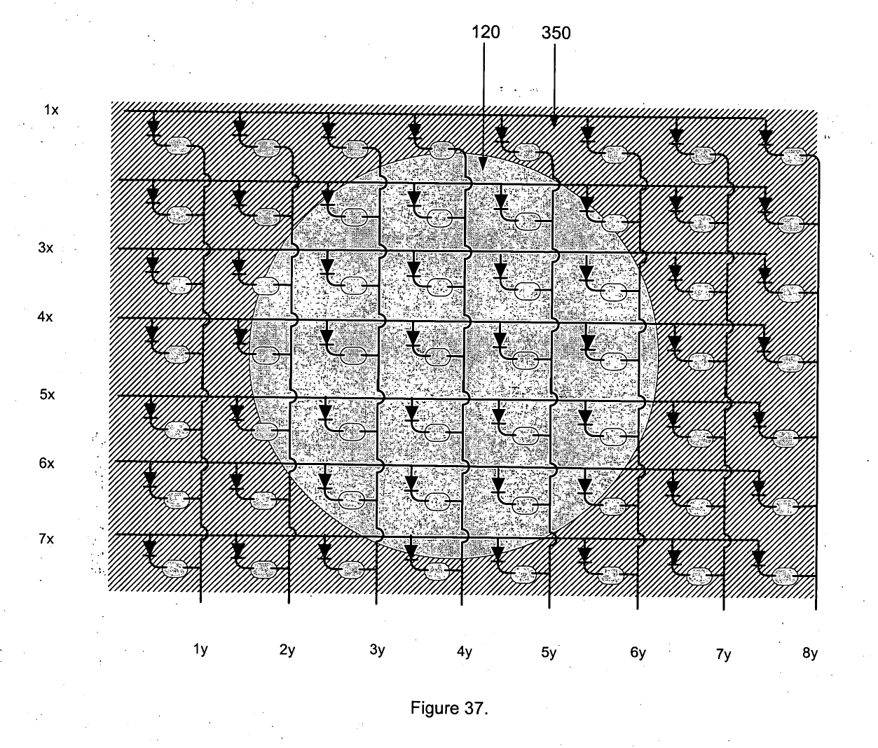

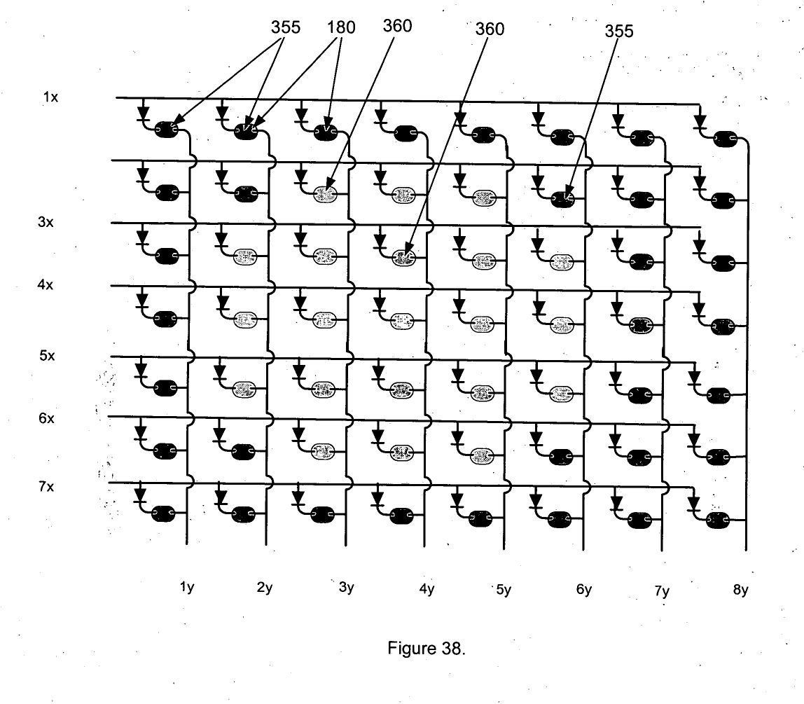

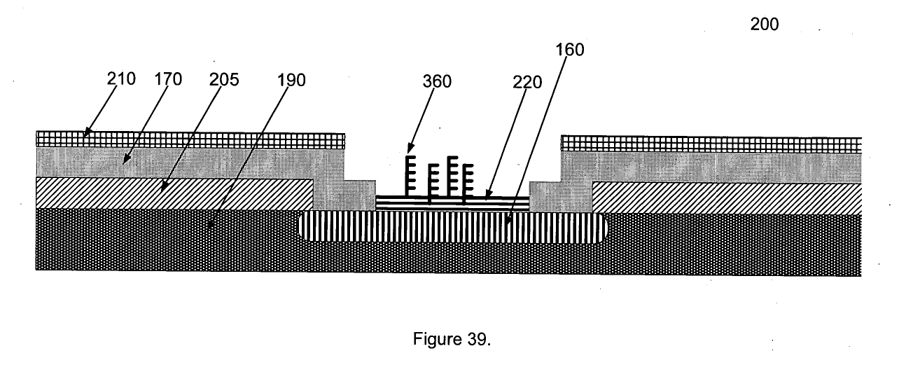

The sense chip as constructed according to the method of the present invention is very versatile and, depending on the type of bioreactive layer, can bind the first of two binding reagents from nucleotide pairs, proteins and ligands, or antigens and antibodies. Here, the use of the chip to sense the presence of known DNA sequences in an unknown solution is presented, although this is not the only, nor necessarily primary, use of the sense chip. A sense chip is either constructed with a metal, metal oxide, or polymer bioreactive layer at the sense gaps or it is treated with a bioreactive substance and the aforementioned bead mop technique results in a sense chip with each sense site containing a bioreactive substance that binds DNA to the surface of the sense gap. The sense chip is positioned in an instrument that will apply, or synthesize, a probe spot of single stranded or double stranded DNA of known sequence via mechanical, ink-jet, or photolithographic application. The location of each probe spot is recorded so that a spot map of the sense chip is created for interpretation of hybridization results. Ideally, each probe spot covers a multitude of sense sites, with 14 or more being the preferred, but not required, number covered in order to compile a statistically meaningful number of data readings. FIG. 36 illustrates multiple sense sites 180 being covered by a single probe spot 120. After allowing the probe spot 120 to dry, a blocking solution 350 is applied to the surface of the sense chip that binds to and deactivates any unreacted sense sites so that future applied target molecules will not bind, FIG. 37. Unbound probe and blocking solution is then rinsed from the sense chip. Alternatively, the rinse solutions may employ the bead mop protocol defined earlier, and as shown in FIG. 26, to ensure that all probe 360 and blocking solution is removed from the surface of the chip and leaving probe 360 and blocking reagent 355 in their respective sense wells, depicted in FIG. 38. The result is all sense sites 180 of the array either being filled with probe or blocking molecules weakly attached to the bioreactive layer of the sense site and covering the sense gap. Probe DNA 360 or blocking substances left in the sense site wells are then exposed to heat and/or UV crosslinking (2600×100 μjoules), described in the literature, to encourage covalent bonding of the probe and/or blocking reagent to their respective sense gap substrates and wells, as depicted in FIG. 39. Following this method, the sense chip 200 is positioned in a test instrument and baseline resistance measurements of each individual sense site are determined and stored. Conversely, the conductive measurements of each sense site can occur either prior to application of the bioreactive layer or after application of the bioreactive layer but before application of probe and blocking material(s). The array is now ready for hybridization.

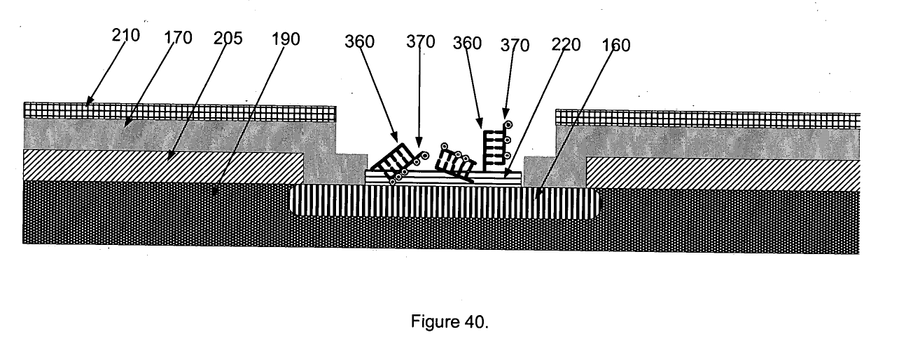

From a sample of interest, biotin labeled cRNA or cDNA is prepared. The biotin labeling may be accomplished chemically or may be incorporated into synthesized copies of the sample template DNA, RNA, or mRNA by substituting biotin labeled ribonucleotides or biotin labeled deoxyribonucleotides as appropriate for a portion of the nucleotides used in a PCR amplification reaction (Molecular Probes, or Sigma Aldrich Catalog). The target solution is then heated to ensure the sample cRNA or cDNA form no secondary structures and is single stranded. If the sense chip probes were double-stranded DNA, then the sense chip is heated to denature the probe. If the DNA was single stranded, no heating is necessary. The target solution then is applied to the surface of the sense chip and allowed to hybridize. The speed of nucleic acid hybridization, and therefore the time required for the hybridization step, varies depending on, but not limited to, the following variables; the length of the probe and target molecules, the melting temperature of the probe with its complementary strand, the temperature of the hybridization reaction, the concentration of probe and target molecules, the G-C content of the probe and target, the salt concentration of the hybridization solution, and the viscosity of the hybridization solution. Many different hybridization protocols exist and are known in the art (See, e.g., Dangler, C., Nucleic Acid Analysis, Principles and BioApplications, 1996; DNA-Microarrays, Botwell, D., Sambrook, J., 2003). During hybridization, target cRNA or cDNA 370 strands that are complementary to probe DNA 360 sequences will bind to one-another, as shown in FIG. 40. After an appropriate amount of time, ranging from 30 minutes to forty-eight hours, the unbound target is rinsed from the sense chip.

A solution containing streptavidin-gold conjugate is then applied to the surface of the sense chip array. FIG. 41 is a simplified drawing showing only the biotin-streptavidin colloidal gold in the sense gap on the bioreactive layer 220. Streptavidin 380 is known to have a high binding affinity for biotin 375 and, after an appropriate incubation time, the biotin-labeled target cRNA or cDNA molecules 370 bound to their respective probes will be linked to streptavidin-gold 390. The streptavidin-gold solution may be procured commercially or produced by the operator using information widely available. (See, e.g., Nanoprobes, Inc., Sigma Aldrich Product Number S 9059). The gold particle attached to the streptavidin can be of various sizes today ranging from 1.4 nm to hundreds of nanometers in diameter. Gold of 10 nm to 50 nm in diameter conjugated to streptavidin seems most prevalent in research, although larger or smaller diameter sizes of gold particles may be used. After an appropriate incubation time the unbound strepatavidin-gold solution is washed from the sense chip array.

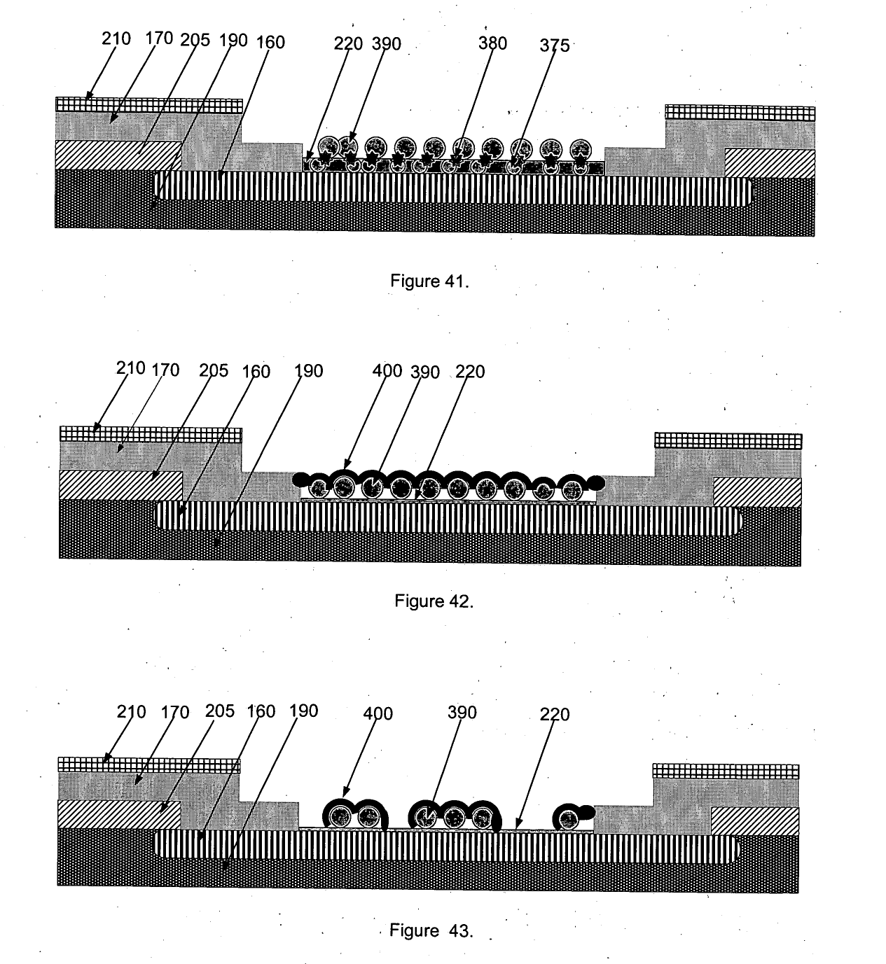

The last processing step is silver enhancement of the bound gold particles. The sense-chip is rinsed with double distilled water to remove chloride ions. A solution of 2M sodium citrate, 0.5M Hydroquinone, and 0.03M silver lactate solution are prepared in a dark room. The solution is then pored onto the sense chip and allowed to react for 2 or 3 minutes. The chip is then rinsed with a 1% acetic acid solution and allowed to incubate for 2 minutes. Lastly the chip is rinsed with fixative solution and allowed to incubate in this solution for 2 minutes. The fixative can be Kodak® Rapid Fix® or prepared solution from Vector Labs. The chip is then rinsed in double distilled water for 5 to 10 minutes and allowed to air dry. The chip is then ready for electrical testing. FIG. 42 depicts colloidal gold particles 390 with a silver coating 400 forming a complete bridge across a sense site gap. FIG. 43 shows colloidal gold particles 390 with a silver coating forming 400 a partial bridge across a sense site gap. Both hybridization results can be detected because of the resulting reduced resistance across the sense site gap.

The sense chip is then placed in a test instrument and the resistance of each sense site is determined. The post hybridization resistance at each sense site is compared to the baseline resistance measurements taken at a point prior to target hybridization to the array (sense chip). A decrease in resistance signifies that hybridization of target molecules took place and the sense site gap has been partially or completely bridged by gold particles coated with silver. At sense sites where no hybridization took place, including all blocked sense sites, no significant change in resistance from the original readings should be noted. If the resistance values of blocked sites has changed, the average of these changes in readings can be considered the noise floor and subtracted from probe sense site conductance readings to give a more accurate reading of true conductance changes at all sense sites. Compiling the baseline probe resistance readings and post-hybridization resistance readings with the physical spot map made earlier, software can be designed to group appropriate neighboring sense site readings into a statistical set of readings for a specific probe. From this, a statistical representation, including confidence levels of the degree of hybridization, can be generated. Successful hybridization and lower resistance means that target molecules complementary to the probe sequence were present in the unknown sample. Similar to techniques currently employed by DNA microarray analysis, known concentrations of a unique reference RNA, DNA or cDNA sequence can be included in the sample. The resistance readings from the various probe/target hybridizations can be compared to the resistance readings from the known reference probe/target concentration sites and quantitative estimates of the amount of target DNA present at each sense site can be determined.

While the above describe procedure outlines single sample hybridizations, the hybridization method may also apply to competitive hybridization procedures. One sample may be non-biotin labeled DNA while a second sample may be biotin labeled. Both samples are then placed in the hybridization mix. A high conductivity reading at a hybridization site would indicate a relative preponderance of the biotin labeled target versus the non-biotin labeled target.

Additionally, the sense site wells of the present invention serve as reaction containers of uniform surface area and volume. Knowing the size of a probe molecule, the number of probe molecules present on the surface of the sense site well can be calculated. Following from this, it is then possible to correlate 100% probe/target hybridization to a maximum current or minimum resistance reading. By this method, increases in current flow or decreases in resistance can be correlated to a quantity of probe and target molecules. Because the construction of the sense site wells has the tight tolerances associated with semiconductor processing techniques, the precision of the quantitative estimates of probe and target using this invention is superior to quantitative estimates based on the free-flowing probe spots of contact spotted microarrays.

The number of processing steps for hybridization and detection of the sense chip is similar to that currently used to process dense microarrays such as those produced by Affymetrix. Unlike current laser scanning, the test instrument that reads the sense chips has no moving parts and is essentially a sensitive, processor-controlled multimeter or electrometer. The resistive sense sites of this invention can be read with low-level DC voltages and currents. As a result, small, reliable, battery operated portable test instruments for the sense chip can be constructed relatively inexpensively. The present invention will allow the precise reading of arrays produced with all 3 popular probe deposition methods. The inexpensive electrical detection instrument of the present invention, when paired with inexpensive probe contact printing instruments, will allow any institution or office to set up a very flexible, microarray analysis capability. The extremely small size of the sense sites even allows state of the art 18 micron diameter photolithographic or inkjet synthesized probe spots to be separated into multiple independent sense sites for reaction and reading. The repetition of independent readings improves confidence in experimental results and the uniform sense size of the site wells allows improved quantification of samples over current methods. The sense chip of the present invention is sensitive, inexpensive, robust, small, repeatable, and intuitively easy to use compared to the present light based scanner detection methods.

### Example of Sense Chip Protocol for the Detection of DNA Sequences in an Unknown Sample Using the Method and Apparatus of the Present Invention.

Sense Chip

1. An electrical sense chip containing thousands of microscopically isolated sense sites, each sense site having its sense gap covered with a bioreactive metal or metal oxide, or treated with a bioreactive layer of amino silane (or other substance such as epoxy silane known to bind to glass and DNA) is placed on a clean level surface.

2. When the probe spotting machine has been set up and just prior to the start of spotting, remove the clear plastic wrap from the top of the sense chip.

Preparation of Stationary Probe Spotting Solution and Spotting of Probe to Sense Chip

1. For each probe spot, place 2 μg of probe DNA in at least 10 μL of a solution containing 10% DMSO and dH₂O. (Significantly less DNA has also been shown to also work).

2. Heat DNA mixture to 95 degrees C. for 15 minutes and then place on ice.

3. Position probe DNA samples in appropriate container (864 well plate) and set into probe spotting machine.

4. Run machine and spot probe onto the sense chip.

5. Allow to air dry and store covered at room temperature.

Blocking and Fixing Stationary DNA to the Sense Chip

1. To a clean 1.5 mL tube add 25 μL of Master mix (0.1 g dextran sulfate, 5 mL formamide, and 1 mL 20×SSC and water up to 7 mL, pH 7.0) and enough fractionated Salmon Sperm DNA to reach a concentration of 250 μg/ML.

2. Heat the mixture to 37 degrees C. and quickly apply to the surface of the sense chip. Cover the sense chip cavity with the supplied plastic cover and place on a slow rocker platform in 37 degrees C. incubator for 30 minutes.

3. Rinse the sense chip twice with 2×SSC solution at 45 degrees C. for 5 minutes.

4. In a separate test tube combine 2 μL of magnetic beads with 23 μL of 2×SSC and apply to the well of the sense chip.

5. Secure the sense chip on a magnetic stirrer base and cover the sense chip with the plastic cover provided. Turn on the magnetic stirring function for 5 minutes.

6. Remove the chip from the platform and quickly rinse in 2×SSC at 45 degrees C. for 5 minutes.

7. Rinse the chip a final time for 5 minutes in 0.1×SSC at 45 degrees C.

8. Let the chip air dry for 10 minutes and then replace plastic cover onto chip. Place the sense chip into the Testing Machine and read the resistance and/or conductance levels of each sense site.

The Sense Chip is Now Ready for Hybridization

Preparing Biotin Labeled Target DNA

1. Take sample DNA and prepare a random primer PCR mix with biotin labeled dUTPs and follow normal PCR procedures. Prepare 25 μL PCR reaction and include dUTP-biotin: dTTP in ratio of 1:3 in the PCR mix.

a. Mix;

| Sample DNA 0.2 μg | approx. 1 | μL |
|---|---|---|
| 2.5 × Random Primer | 10 | μL |
| Water | 9.8 | μL |

b. Denature the mixture in a PCR machine at 100 degrees C. for 10 minutes.

c. Add;

| | |
|---|---|
| dNTPs | 2.5 uL |
| Biotin Labeled dUTP | 1.0 μL |
| Klenow Fragment | 0.7 μL |
| Total | 25 μL |

d. Mix well and incubate for 4 hours at 37 degrees C.

2. Upon completion run a 1% Agarose gel and DNA ladder to determine if the biotin labeled target sequences have been evenly generated. The biotin labeled sample should show as a smear with the darkest areas between 200 bp and 800 bp.

3. Using an Amersham MicroSpin G-50 column, snap open the columns and place in a new 1.5 mL tube and spin at 770 rcf for 1 minute to remove excess buffer. Discard the buffer and replace the column into the 1.5 mL tube. Apply the biotin labeled target solution and spin again at 770 rcf for 2 minutes. Discard the column and place the tube with target DNA on ice.

The Target is Ready for Hybridization.

Preparing Hybridization Mixture

1. Combine 25 μL of the target DNA mixture with 25 μL (2 μg) of Cot 1 DNA in a 1.5 mL tube and ethanol precipitate.
a. Ethanol Precipitation
Combine 2.5 volumes of ice cold ethanol (100%) and 0.1 volume of 3M sodium acetate pH 5.2, with 25 μL of target DNA.
b. Spin at 14,000 rpm for 30 minutes at 4 degrees C.
c. Decant supernatant and air dry the pellet for 10 minutes. Use a rolled Kimwipe to remove any excess ethanol.

2. Resuspend the Target pellet in 5 μL water, 10 μL 20% SDS, and 35 μL of Mastermix.

Mastermix;
Add 0.1 gram dextran sulfate (mw approximately 500,000), 5 mL of formamide, 1 mL 20×SSC, and water up to 7 mL at pH 7.0.

3. After the pellet is resupended, denature the target mixture at 75 degrees C. for 15 minutes.

4. Remove the target mixture to a 37 degrees C. incubator and allow the Cot 1 DNA to preanneal to the target for a minimum of 1 hour. This will block repetitive sequences present in the target DNA.

Hybridization of Target to the Sense Chip

1. Rinse sense chip in 2×SSC for 5 minutes at 45 degrees C. Air dry for 5 minutes.

2. Place the sense chip in a Stratalinker UV machine and apply 2600×100 μjoules to covalently link the Probe DNA to the surface of the sense gaps.

3. Take the warm target mixture and apply to the surface. Seal the sense chip into the plastic hybridization chamber provided. Place the assembly on a slow rocker platform in an incubation oven at the researchers prescribed temperature and length of time.

Washing the Sense Chip

1. Pre-warm the wash solutions: wash 1 (50% formamide and 50% 1×SSC); wash 2 (2×SSC); wash 3 (0.1×SSC); and wash 4 (PN Buffer 0.1M sodium phosphate with 0.1% NP-40) to 45 degrees C. in large Coplin jars.

2. Rinse/soak the array chip in above rinse solutions for;

| | |
|---|---|
| Formamide | 10 minutes |
| 2 × SSC | 10 minutes |
| 0.1 × SSC | 5 minutes |
| PN Buffer | 5 minutes |

3. Shake the last liquid from the Sense chip well.

Application of the Streptavidin-Gold Conjugate

1. Prepare 25 μL Streptavidin/Gold solution in amber tubes. 1:4 dilution of Streptavidin Gold into 15 mM NaCl solution and 0.1% BSA.

2. Pipet the solution onto the Sense chip and cover with the plastic chip cover.

3. Place the Sense chip on a slow rocker in an Incubator at 37 degrees C. for 30 minutes.

4. Rinse with 1×SSC for 1 minute, then rinse with 0.1×SSC for 1 minute.

5. Air dry for 15 minutes.

Silver Enhancement of Gold Particles and Measuring the Sense Chip

1. The Sense chip is rinsed in double distilled water at 45 degrees C. for 1.5 minutes to remove sodium ions.

2. In a dark room prepare a 100 μL solution of 2M sodium citrate, 0.5M Hydroquinone, and 0.03M silver lactate solution. Pour 30 μL of the solution onto the sense chip and let incubate at room temperature for 3 minutes.

3. Rinse the sense chip with 1% acetic acid solution and allow it to incubate in 30 μL of this solution for 2 minutes.

4. Rinse the sense chip with Kodak® Rapid Fix® fixative solution and incubate in 30 μL of this solution for 3 minutes at room temperature.

5. Rinse the sense chip in double distilled water for 1.5 minutes at room temperature, then for 3 minutes in 0.1% SSC at room temperature for 3 minutes. A last room temperature wash in double distilled water for 30 seconds completes the silver enhancement.

6. Allow the sense chip to air dry or place it on a heat block at 37 degrees C. and dry. The sense chip is ready to be measured.

Decreases in the resistivity of the sense sites at this point, compared with the original resistance readings made prior to hybridization of the target, indicates successful probe/target hybridization.

While the invention has been described above by reference to various embodiments, it will be understood that changes and modifications may be made without departing from the scope of the invention, which is to be defined only by the appended claims and their equivalents.

1. An improved apparatus for identification of biomolecules comprising:
a planar semiconductor substrate having a top side, a bottom side, and external electrical contacts;
a plurality of sense sites formed within the substrate top side further defining a matrix of sense sites, wherein each sense site comprises a four sided well constructed from silicon dioxide isolation layers, a gap to receive probe molecules, a gap substrate capable of changing its resistivity with appropriate processing serving as the bottom of the four sided well, means to affix probe molecules in the sense site gap to the gap substrate, means to electrically detect biomolecules within the gap, means to vary base resistivity across the sense site gap, and means to break surface tension of sample liquids;
means to control the semiconductor substrate temperature;
means to protect the semiconductor substrate top side;
means to sequentially read parallel electrical measurements from multiple sense sites and correlate output data between all sense sites allowing competitive hybridization, chip-to-standard, or chip-to-chip baseline measurements;
means to separate a spot sample-applied-to gaps in the matrix from nearby sense sites not in contact with the sample; and
means to separate a spot sample into multiple, separate sense site wells.

2. The apparatus of claim 1, wherein the substrate is silicon.

3. The apparatus of claim 1, wherein the substrate is germanium.

4. The apparatus of claim 1, wherein means to affix probe molecules in the sense site gap to the gap substrate further comprises a bioreactive material selected from carbon, hydrophillic organic polymers, inorganic metal oxides and inorganic metal nitrides.

5. The apparatus of claim 1, wherein the substrate further comprises a diode or unidirectional semiconductor device.

6. The apparatus of claim 1, wherein means to electrically detect biomolecules within the gap further comprises two conductive traces located on opposite sides of the sense site gap, wherein the variable resistance gap substrate separates the conductive traces and is connected to the conductive traces, a unidirectional electrical element in series with one of the two conductive traces, wherein the plurality of sense sites forms a two dimensional matrix on the substrate top side which, except for the sense site addressed, energizes the sense site electrical elements in a reverse biased mode, and wherein each sense site is addressable and reliably read using microprocessors, microcontrollers, multiplexers, demultiplexers, addressable switches, temperature sensitive elements, and amplifier circuits produced on the substrate.

7. The apparatus of claim 1, wherein means to affix probe molecules in the sense site gap to the gap substrate further comprises a functionalizing reagent applied to the gap substrate.

8. The apparatus of claim 1, wherein means to protect the substrate top side further comprises a passivation layer coating disposed on the substrate top side.

9. The apparatus of claim 1, wherein means to separate a spot sample applied to gaps in the matrix from sense sites not in contact with the sample and means to separate a spot sample into multiple, separate sense site wells further comprise at least one metallic or magnetic bead of sufficient diameter moving on the substrate top side so that the bead cannot enter or drop down into the sense site well, and wherein means for movement of the bead on the substrate top side consists of substrate motion, external permanent magnets or electromagnets, or a varying external magnetic field.

10. The apparatus of claim 1, wherein means to break surface tension of sample liquids further comprises at least one liquid shunt.

11. The apparatus of claim 1, wherein means to control substrate temperature further comprises a temperature sensitive diode connected to the external contacts of the substrate.

12. The apparatus of claim 6, wherein the conductive traces further comprise rounded edges.

13. The apparatus of claim 6, wherein the gap substrate further comprises non-oxidized semiconductor material.

14. The apparatus of claim 1, wherein means to sequentially read parallel electrical measurements from multiple sense sites and correlate output data between all sense sites allowing competitive hybridization, chip-to-standard, or chip-to-chip-baseline measurements comprises a portable or plug-in test instrument.

15. The apparatus of claim 14, wherein means to sequentially read parallel electrical measurements from multiple sense sites and correlate output data between all sense sites allowing competitive hybridization, chip-to-standard, or chip-to-chip baseline measurements further comprises a portable analysis instrument comprising a chamber to perform liquid processing of the array/sense site and means to read, store, and transmit measurements of sense site resistance or conductance.

16. The apparatus of claim 15, wherein means to sequentially read parallel electrical measurements from multiple sense sites and correlate output data between all sense sites allowing competitive hybridization, chip-to-standard, or chip-to-chip baseline measurements further comprises a fast comparator circuit that will signal when potentially damaging high currents are present on the chip and disconnect and protect the sense chip from the fault.

17. The apparatus of claim 1, wherein spacing of sense sites is not more than 50 microns from the center of a sense site gap to the center of the next closest sense site gap in any direction.

18. The apparatus of claim 1, wherein at least three neighboring sense sites-each contain sample aliquots from the same, single, mechanical application or synthetic construction of a probe spot in the region, and wherein each sense site analyses its respective aliquot independently of all other sense sites.

19. The apparatus of claim 1, wherein the substrate further comprises a programmable fuse link circuit which allows the mean circuit resistance to be adjusted to specific data sheet values.

20. The apparatus of claim 1, wherein the substrate further comprises a temperature sensitive element such as a diode which produces a signal characterized to determine and control substrate temperature.

21. The apparatus of claim 1, wherein the substrate further comprises a matrix of closed, conductive sense sites for parametric testing and wherein which, during manufacture, the closed sense sites are opened using laser trimming techniques to preserve the substrate's unidirectional diode matrix after testing.

22. A method for identification of biomolecules employing probe spot application to a semiconductor sense chip substrate having a top side and comprising a plurality of sense sites formed within the substrate top side as an array surface with each sense site further comprising an amino-silane or metal oxide sense site well having a predetermined and uniform width, length, and depth dimension and a sense site substrate, the method comprising the steps of:
a. denaturing probe spot DNA by heating to 95 degrees C. for 15 minutes;
b. applying all probe spots to the array surface and drying;
c. coating the array surface with salmon sperm solution;
d. allowing the coated array surface to stand for a predetermined period of time;
e. rinsing the coated array surface with 2×SSC;
f. preparing a solution of 2×SSC and latex coated beads with a diameter at least 10× larger than the sense site well length;
g. placing the substrate on a magnetic base instrument;
h. coating the substrate top surface with the bead solution;
i. switching on the magnetic base instrument;
j. allowing the substrate to be treated by the magnetic base instrument for a predetermined period of time;
k. immediately rinsing the substrate top surface with 2×SSC twice at room temperature for a predetermined period of time for each wash;
l. allowing the substrate top side to dry while being stored in a covered environment;
m. applying means to establish covalent bonds to the sense site substrate;
n. taking baseline electrical measurements; and
o. covering and storing the substrate until ready for hybridization.

23. The method of claim 22, wherein the means to establish covalent bonds to the sense site substrate further comprises the step of baking the substrate at 80 degrees centigrade for 80 minutes.

24. The method of claim 22, wherein the means to establish covalent bonds to the sense site substrate further comprises the step of applying UV energy using a UV crosslinker.

25. A method for identification of biomolecules employing probe spot application to a semiconductor sense chip substrate having a top side and comprising a plurality of sense sites formed within the substrate top side as an array surface with each sense site further comprising an amino-silane or metal oxide sense site well having a predetermined and uniform width, length, and depth dimension and a sense site substrate, the method comprising the steps of:
a. preparing a stationary probe spotting solution;
b. spotting the stationary probe to the sense chip;
c. fixing stationary DNA to and blocking the sense chip;
d. preparing biotin labeled target DNA;
e. preparing a hybridization mixture;
f. hybridization of target to the sense chip;
g. washing the sense chip;
h. applying streptavidin-gold conjugate;
i. enhancing the streptavidin-gold conjugate with silver; and
j. measuring the sense chip.

26. The method of claim 25, wherein the steps of preparing a stationary probe spotting solution and spotting the stationary probe to the sense chip further comprise the sub-steps of:
a. placing 2 μg of probe DNA in at least 10 μL of a solution containing 10% DMSO and dH₂O for each probe spot;
b. heating the DNA mixture to 95 degrees centigrade for 15 minutes and then placing the mixture on ice;
c. positioning the probe DNA samples in an appropriate container (864 well plate) and setting the container into a probe spotting machine;
d. operating machine and spotting each probe onto a separate sense chip; and
e. allowing each spotted sense chip to air dry and storing the dried, spotted sense chips in a covered environment at room temperature.

27. The method of claim 25, wherein the step of blocking and fixing stationary DNA to the sense chip further comprises the sub-steps of:
a. to a clean 1.5 mL tube, adding 25 μL of Master mix (0.1 g dextran sulfate, 5 mL formamide, and 1 mL 20×SSC and water up to 7 mL, pH 7.0) and enough fractionated Salmon Sperm DNA to reach a concentration of 250 μg/mL;
b. heating the mixture to 37 degrees centigrade and quickly applying it to the-surface of the sense chip, covering the sense chip cavity with a supplied plastic cover, and placing the sense chip on a slow rocker platform in 37 degrees centigrade incubator for 30 minutes;
c. rinsing the sense chip twice with 2×SSC solution at 45 degrees centigrade for 5 minutes;.
d. in a separate test tube, combining 2 μL of magnetic beads with 23 μL of 2×SSC and applying the combination to the well of the sense chip;
e. securing the sense chip on a magnetic stirrer base and covering the sense chip with the plastic cover provided while the magnetic stirring function is applied to the sense chip for 5 minutes;
f. removing the sense chip from the platform and quickly rinsing the sense chip in 2×SSC at 45 degrees centigrade for 5 minutes;
g. rinsing the sense chip a final time for 5 minutes in 0.×SSC at 45 degrees centigrade;
h. allowing the sense chip to air dry for 10 minutes and, if desired, replacing plastic cover onto the sense chip; and
i. placing the sense chip into a testing machine and reading the resistance and/or conductance levels of each sense site.

28. The method of claim 25, wherein the step of preparing Biotin labeled target DNA further comprises the sub-steps of:
a. taking sample DNA and preparing a random primer PCR mix with biotin labeled dUTPs, following normal PCR procedures wherein the random primer PCR mix comprises 25 μL PCR reaction and includes dUTP-biotin: dTTP in ratio of 1:3 in the PCR mix, which follows the sub-steps of (i) mixing 0.2 μg of sample DNA (approx. 1 μL), 10 μL of 2.5× Random Primer, and 9.8 μL water, (ii) denaturing the mixture in a PCR machine at 100 degrees centigrade for 10 minutes, (iii) adding 2.5 μL dNTPs, 1.0 μL Biotin labeled dUTP, and 0.7 μL Klenow Fragment for an approximate total volume of 25 μL, and (iv) mixing well and incubating for 4 hours at 37 degrees centigrade;
b. upon completion, running a 1% Agarose gel and DNA ladder to determine if the biotin labeled target sequences have been evenly generated wherein the biotin sample should show a smear with the darkest areas between 200 bp and 800 bp;
c. using an Amersham MicroSpin G-50 column, snapping open the columns and placing in a new 1.5 mL tube;
d. spining at 770 rcf for 1 minute to remove excess buffer;
e. discarding the buffer and replacing the column into the 1.5 mL tube;
f. applying the biotin labeled target solution and spinning again at 770 rcf for 2 minutes; and
g. discarding the column and placing the tube with target DNA on ice.

29. The method of claim 25, wherein the step of preparing a hybridization mixture comprises the sub-steps of:
a. combining 25 μL of the Target DNA mixture with 25 μL (2 μg) of Cot 1 DNA in a 1.5 mL tube and ethanol precipitate, which further comprises 2.5 volumes of ice cold ethanol (100%) and 0.1 volume of 3M sodium acetate pH 5.2, with 25 μL of Target DNA;
b. spining the combination at 14,000 rpm for 30 minutes at 4 degrees centigrade;
c. decanting the supernatant and air drying the pellet for 10 minutes, using a rolled Kimwipe to remove any excess Ethanol;
d. resuspending the Target pellet in 5 μL water, 10 μL 20% SDS, and 35 μL of Mastermix, which further comprises 0.1 gram dextran sulfate (mw approximately 500,000), 5 mL of formamide, 1 mL 20×SSC, and water up to 7 mL at pH 7.0;
e. after the pellet is resupended, denaturing the Target mixture at 75 degrees centigrade for 15 minutes; and
f. removing the Target mixture to a 37 degrees centigrade incubator and allowing the Cot 1 DNA to preanneal to the Target for a minimum of 1 hour, thus blocking repetitive sequences in the target.

30. The method of claim 25, wherein the step of hybridization of target to the sense chip comprises the sub-steps of:
a. rinsing the sense chip in 2×SSC for 5 minutes at 45 degrees centigrade and air drying the rinsed sense chip for 5 minutes;
b. placing the sense chip into a Stratalinker UV machine and applying 2600×100 μjoules to covalently link the Probe DNA to the surface of the sense gaps;
c. applying the warm target mixture to the sense chip surface;
d. sealing the sense chip into a plastic hybridization chamber provided;
e. placing the assembly on a slow rocker platform in an incubation oven at the researcher's prescribed temperature and for a predetermine period of time.

31. The method of claim 25, wherein the step of washing the sense chip comprises the sub-steps of:
a. pre-warming the wash solutions of wash 1 (50% formamide and 50% 1×SSC), wash 2 (2×SSC), wash 3 (0.1×SSC), and wash 4 (PN Buffer 0.1M sodium phosphate with 0.1% NP-40) to 45 degrees centigrade in large Coplin jars;
b. Rinsing/soaking the array sense chip in above rinse solutions for the following time periods for each solution (i) Formamide for 10 minutes, (ii) 2×SSC for 10 minutes, (iii) 0.1×SSC for 5 minutes, and (iv)PN Buffer for 5 minutes;
c. shaking the last remaining liquid from the sense chip well and allowing the sense chip to air dry for 15 minutes.

32. The method of claim 25, wherein the step of applying streptavidin-gold conjugate comprises the sub-steps of:
a. preparing a 25 μL streptavidin/gold solution in amber tubes comprising a 1:4 dilution of streptavidin gold into 15 mM NaCl solution and 0.1% BSA;
b. pipetting the streptavidin/gold solution onto the sense chip and covering the sense chip with the plastic sense chip cover;
c. placing the sense chip on a slow rocker in an incubator at 37 degrees centigrade for 30 minutes;
d. rinsing the sense chip with 1×SSC for 1 minute;
e. rinsing the sense chip with 0.1×SSC for 1 minute; and
f. allowing the sense chip to air dry for 15 minutes.

33. The method of claim 25, wherein the step of enhancing the streptavidin-gold conjugate with silver comprises the sub-steps of:
a. rinsing the sense chip in double distilled water at 45 degrees centigrade for 1.5 minutes to remove sodium ions;
b. preparing a 100 μL solution of 2M sodium citrate, 0.5M hydroquinone, and 0.03M silver lactate solution in a dark room, and pouring 30 μL of the prepared solution onto the sense chip and letting the treated sense chip incubate at room temperature for 3 minutes;
c. rinsing the sense chip with 1% acetic acid solution and allowing the treated sense chip to incubate in 30 μL of this solution for 2 minutes;
d. rinsing the sense chip with Kodak® Rapid Fix® fixative solution and allowing the treated sense chip to incubate in 30 μL of this solution for 3 minutes at room temperature;
e. rinsing the sense chip in double distilled water for 1.5 minutes at room temperature, then rinsing the sense chip for 3 minutes in 0.1% SSC at room temperature for 3 minutes;
f. washing the sense chip in double distilled water for 30 seconds at room temperature wash to complete the silver enhancement, and
g. allowing the silver enhanced sense chip to dry.

34. The method of claim 25, wherein the step of measuring the sense chip shows probe/target hybridization to be successful when deceased resistivity of the sense sites after enhancing the streptavidin-gold conjugate with silver compared with the original resistance readings made prior to hybridization of the target.

35. The apparatus of claim 1, wherein the semiconductor substrate comprises various resistive sense sites.

36. The apparatus of claim 1, wherein a plurality of semiconductor substrates comprise a substrate family of various resistive sense sites.
